(19) United States
(12) Patent Application Publication
(10) Pub. No.: US 2022/0380310 A1
(43) Pub. Date: Dec. 1, 2022

(54) P2X7R ANTAGONISTS

(71) Applicants: Ligang QIAN, San Diego, CA (US); Chunbo ZHANG, Jurupa Valley, CA (US)

(72) Inventors: Ligang QIAN, San Diego, CA (US); Chunbo ZHANG, Jurupa Valley, CA (US)

(21) Appl. No.: 17/624,348

(22) PCT Filed: Jun. 29, 2020
(86) PCT No.: PCT/US2020/040076

§ 371 Date: Jan. 2, 2022

Related U.S. Application Data
(60) Provisional application No. 62/869,040, filed on Jul. 1, 2019.

(51) Int. Cl. C07D 211/96; C07D 401/12; C07D 413/12; C07D 405/12; C07D 211/56; C07D 241/06

(52) U.S. Cl. CPC C07D 211/96; C07D 401/12; C07D 413/12; C07D 405/12; C07D 211/56; C07D 241/06

(57) ABSTRACT

A compound has Formula I:

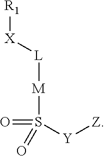

R₁ is hydrogen, hydroxy, halogen, nitro, amino, alkyl, alkoxy, alkylamino, cycloalkyl, cycloalkyamino, heterocyclyl, aryl, heteroaryl, —NR₇R₈, —CO—R₁₀, or —NH—CO—R₁₀; L is a bond, a heterocyclic bivalent group, a heteroaromatic bivalent group, or an aromatic bivalent group; M is a bond, alkyl, aryl, heterocyclic bivalent group, heteroaromatic bivalent group, or aromatic bivalent group; X is a bond, —O—, —S—, —SO₂—, —CO—, —NR₉—, —(CH₂)ₘ—, or heterocyclic bivalent group, m is 1, 2, 3, 4, 5, or 6; Y is a bond, —NH—, heterocyclic bivalent group, heteroaromatic bivalent group, bivalent benzyl group, or aromatic bivalent group; and Z is hydrogen, halogen, alkyl, aryl, heterocyclyl, heteroaryl, —NR₇R₈, —CO—R₁₀, or —NH—CO—R₁₀; R₇, R₈, and R₉ are independently hydrogen, hydroxy, halogen, nitro, amino, alkyl, alkoxy, alkylamino, cycloalkyl, cycloalkyamino, heterocyclyl, or heteroaryl; and R₁₀ is —O-tert-butyl, —CH₂CH₂-phenyl, hydrogen, hydroxy, halogen, nitro, amino, alkyl, alkoxy, alkylamino, cycloalkyl, cycloalkyamino, heterocyclyl, or heteroaryl.

This application claims priority to U.S. Provisional Application No. 62/869,040, filed on Jul. 1, 2019, which is incorporated by reference for all purposes as if fully set forth herein.

### FIELD OF THE INVENTION

The present invention relates to medical chemistry, particularly, purinergic receptor P2X7 (P2X7R) antagonists and their use in medicine and diagnostics.

### BACKGROUND OF THE INVENTION

P2X7R is a member of ligand-gated ion channel and is expressed in a variety of cell types. It plays a crucial role in development and in normal physiological activity. Compared to other P2X receptors, P2X7R displays the most prominent role in various pathological conditions since it is a key player in inflammatory and immune process. Its expression in macrophages and monocytes in the periphery and especially its expression in glial cells (microglia, astrocytes, oligodendrocytes, and Schwann cells) in the nervous system make them a therapeutic target for neurodegenerative diseases and other neuropathological conditions. P2X7R is also expressed in antigen-presenting cells, keratinocytes, salivary acinar cells, hepatocytes, erythrocytes, erythroleukaemic cells, monocytes, fibroblasts, bone marrow cells, neurons, and renal mesangial cells. In the brain, areas of high P2X7R expression were found in the anterior olfactory nucleus, cerebral cortex, piriform cortex, lateral septal nucleus, hippocampal pyramidal cell layers (CA1, CA3, and CA4), pontine nuclei, external cuneate nucleus, and medial vestibular nucleus. P2X7R messenger RNA hybridization signals were also observed in motor neurons of the trigeminal motor nucleus, facial nucleus, hypoglossal nucleus, and the anterior horn of the spinal cord. Studies have shown that P2X7R acts as scavenger receptors that promote phagocytosis by directly binding apoptotic corpses and foreign debris to their extracellular domain in the absence of extracellular adenosine triphosphate (ATP). P2X7R differs from other P2X receptors because a high concentration of ATP is required to activate it, which is consistent with the involvement of P2X7R in various pathological conditions. Upon activation by agonists, the ion channels formed by P2X7R may be permeable only to small ions initially, but move slowly to a state that is permeable to both small and large molecules under the continued presence of agonists. Prolonged large pore opening of P2X7R channel leads to the release of proinflammatory cytokines such as IL-1β, IL-6, IL-18, chemokine CCL2, and/or tumor necrosis factor TNFα, which promotes inflammation and may regulate additional events leading to cell deterioration or even death. Inhibition of P2X7R may offer novel perspectives for anti-inflammatory therapy.

Accumulating data have indicated that P2X7R is a key player in inflammation. Inhibition of P2X7R activity by agents or making P2X7R deficient by gene deletions, gene mutations or gene silences can improve various pathological conditions mediated by P2X7R. Compounds of the present invention or a pharmaceutically acceptable salt thereof for the treatment of humans and lower animals may be applied in established symptoms and prophylactic treatments in P2X7R mediated conditions, including but not limited to, abnormal platelet function diseases, addition, bone diseases, cancers, cardiovascular diseases, depression, diabetes, fever, gastrointestinal dysfunction, inflammation and inflammatory conditions, immunological diseases, impotence or erectile dysfunction, kidney dysfunction, liver dysfunction, neurodegenerative diseases and other neuropathological conditions, and pain and pain associated disorders. Neuropathological conditions such as Alzheimer's disease, amyotrophic lateral sclerosis, Huntington's disease, Parkinson's disease, spinal cord injury, cerebral ischemia, head trauma, meningitis, sleep disorders, mood and anxiety disorders, epilepsy, HIV-induced neuroinflammation and CNS damage, chronic neuropathic and inflammatory pain, and peripheral inflammatory disorders and autoimmune diseases including age-related macular degeneration, airways hyper-responsiveness, allergic dermatitis, asthma, atherosclerosis, bronchitis, burn injury, chronic obstructive pulmonary disease, Crohn's disease, diabetes, fatty liver disease, fibrosis, glomerulonephritis, growth and metastasis of malignant cells, irritable bowel syndrome, ischemic heart disease, liver fibrosis, lung emphysema, muscular dystrophy, myoblastic leukaemia, osteoporosis, ostheoarthritis, psoriasis, rheumatoid arthritis, septic shock, Sjogren's syndrome, skin injury, and ulcerative colitis are all examples where the involvement of P2X7R has been implicated. Due to the importance of P2X7R in human health, invention of new P2X7R antagonists represents an attractive avenue for new therapeutic agent development. Although P2X7R antagonists are described in various patent applications, there are needs for new P2X7R antagonists that are effective and can be delivered into the different target organs whose pathology is mediated by P2X7R. P2X7R antagonists also exhibit promising potentials in medical diagnostics or targeted drug delivery. A number of ¹¹C and ¹⁸F radioisotope labeled P2X7R antagonists are applied as the positron emission tomography (PET) imaging tracers to elucidate the locations and expression of P2X7R in the nervous system or in other lesion areas. Developing P2X7R antagonists for diagnosis and treatments of various diseases represents an attractive forward looking perspective.

### SUMMARY OF THE INVENTION

In one embodiment, the present invention provides a compound having the following Formula I:

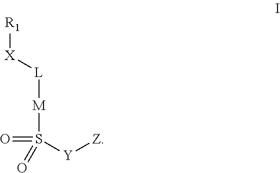

In Formula I, R₁ is hydrogen, hydroxy, halogen, nitro, amino, alkyl, alkoxy, alkylamino, cycloalkyl, cycloalkyamino, heterocyclyl, aryl, heteroaryl, —NR₇R₈, —CO—R₁₀, or —NH—CO—R₁₀; L is a bond, a heterocyclic bivalent group, a heteroaromatic bivalent group, or an aromatic bivalent group; M is a bond, alkyl, aryl, a heterocyclic bivalent group, a heteroaromatic bivalent group, or an aromatic bivalent group; X is a bond, —O—, —S—, —SO₂—, —CO—, —NR₉—, —(CH₂)ₘ—, or a heterocyclic bivalent group, m is 1, 2, 3, 4, 5, or 6; Y is a bond, —NH—, a heterocyclic bivalent group, a heteroaromatic bivalent group, a bivalent benzyl group, or an aromatic bivalent group; Z is hydrogen, halogen, alkyl, aryl, heterocyclyl, heteroaryl, —NR₇R₈, —CO—R₁₀, or —NH—CO—R₁₀; R₇, R₈, and R₉ are independently hydrogen, hydroxy, halogen, nitro, amino, alkyl, alkoxy, alkylamino, cycloalkyl, cycloalkyamino, heterocyclyl, or heteroaryl; and R₁₀ is —O-tert-butyl, —CH₂CH₂-phenyl, hydrogen, hydroxy, halogen, nitro, amino, alkyl, alkoxy, alkylamino, cycloalkyl, cycloalkyamino, heterocyclyl, or heteroaryl; an isomer thereof, a tautomer thereof, a pharmaceutical acceptable solvate thereof, or a pharmaceutical acceptable prodrug thereof.

In another embodiment, in Formula I, L is

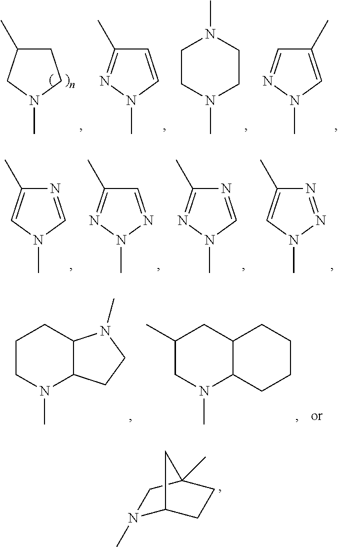

and n is 0, 1, 2, 3, 4, or 5.

In another embodiment, in Formula I, Y is a bivalent phenyl group, a bivalent naphthyl group, a bivalent quinolinyl group, or a bivalent isoquinolinyl.

In another embodiment, M in Formula I is a bond and the compound has the following Formula II:

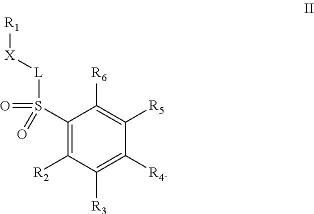

R₁ is hydrogen, hydroxy, halogen, nitro, amino, alkyl, alkoxy, alkylamino, cycloalkyl, cycloalkyamino, heterocyclyl, aryl, heteroaryl, —NR₇R₈, —CO—R₁₀, or —NH—CO—R₁₀; R₂, R₃, R₄, R₅, and R₆ are independently hydrogen, hydroxy, halogen, nitro, amino, alkyl, alkoxy, alkylamino, cycloalkyl, cycloalkyamino, heterocyclyl, or heteroaryl; L is a bond, a heterocyclic bivalent group, a heteroaromatic bivalent group, or an aromatic bivalent group; X is a bond, —O—, —S—, —SO₂—, —CO—, —NR₉—, or —(CH₂)ₘ—, m is 1, 2, 3, 4, 5, or 6; R₇, R₈, and R₉ are independently hydrogen, hydroxy, halogen, nitro, amino, alkyl, alkoxy, alkylamino, cycloalkyl, cycloalkyamino, heterocyclyl, or heteroaryl; and R₁₀ is —O-tert-butyl, —CH₂CH₂-phenyl, hydrogen, hydroxy, halogen, nitro, amino, alkyl, alkoxy, alkylamino, cycloalkyl, cycloalkyamino, heterocyclyl, or heteroaryl.

In another embodiment, in Formula II, L is

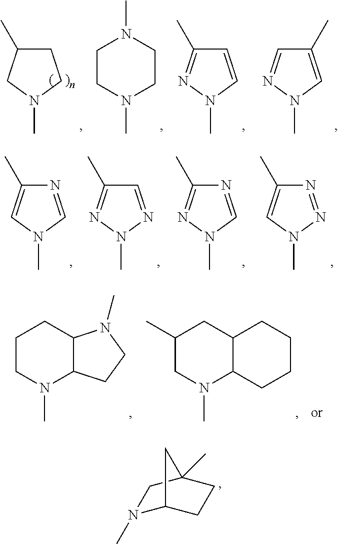

and n is 0, 1, 2, 3, 4, or 5.

In another embodiment, in Formula II, R₂, R₃, R₄, R₅, and R₆ are independently hydrogen, hydroxy, halogen, nitro, amino, alkyl, alkoxy, alkylamino, cycloalkyl, cycloalkyamino, heteroaryl,

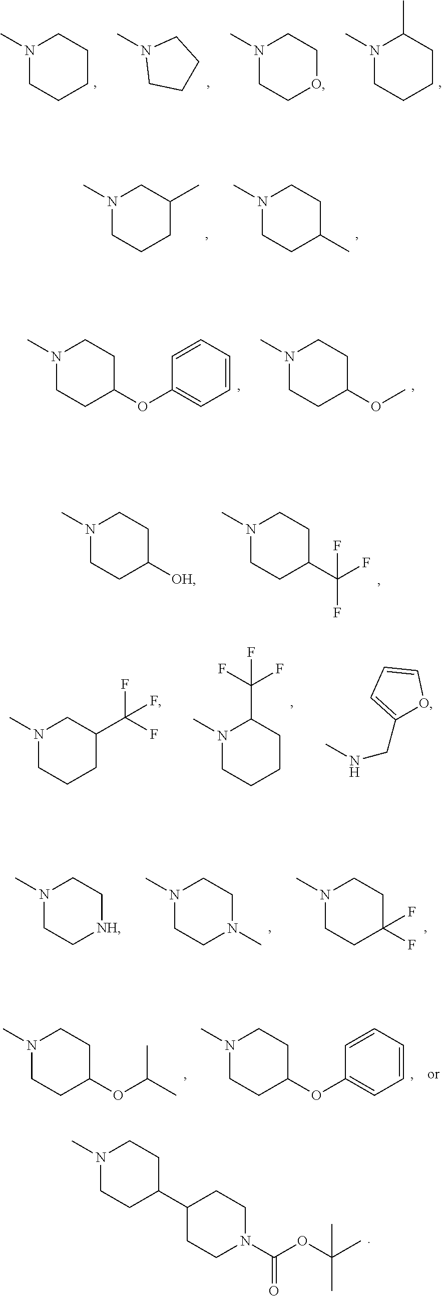

In another embodiment, in Formula II, the compound is selected from the group consisting of:

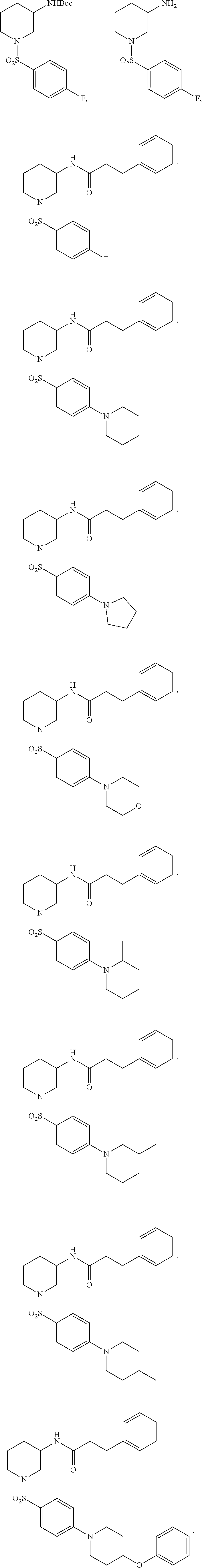

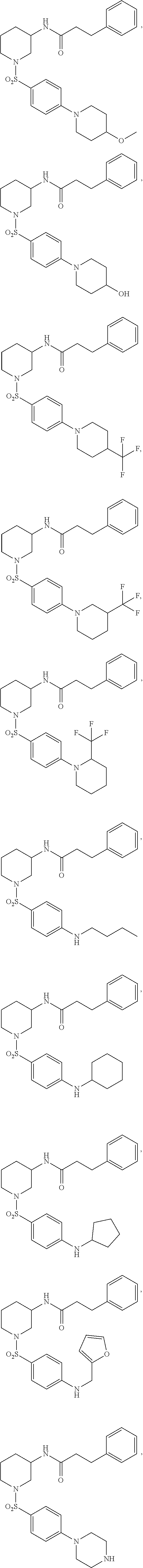

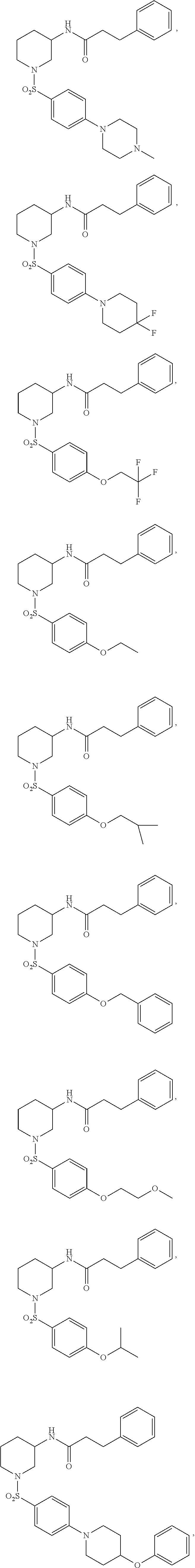

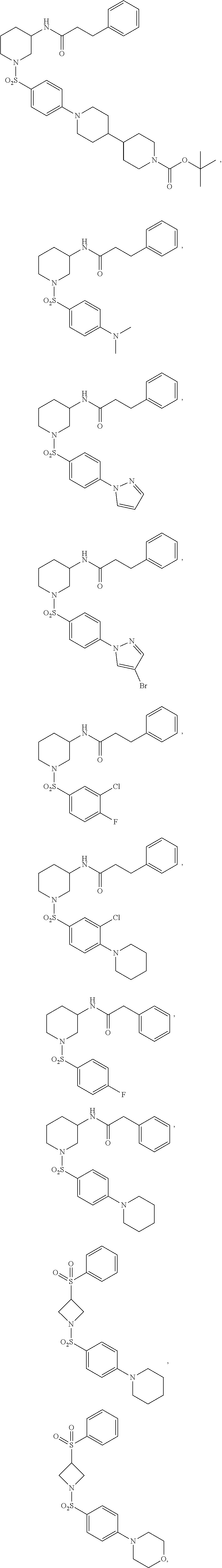

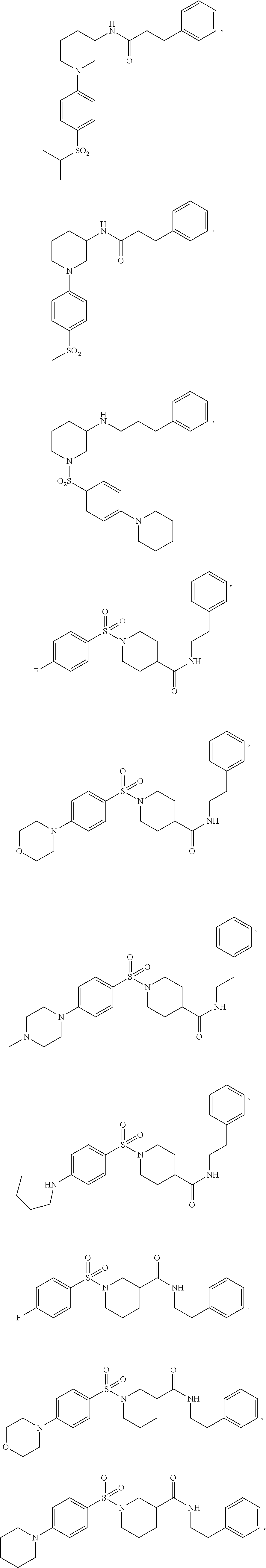

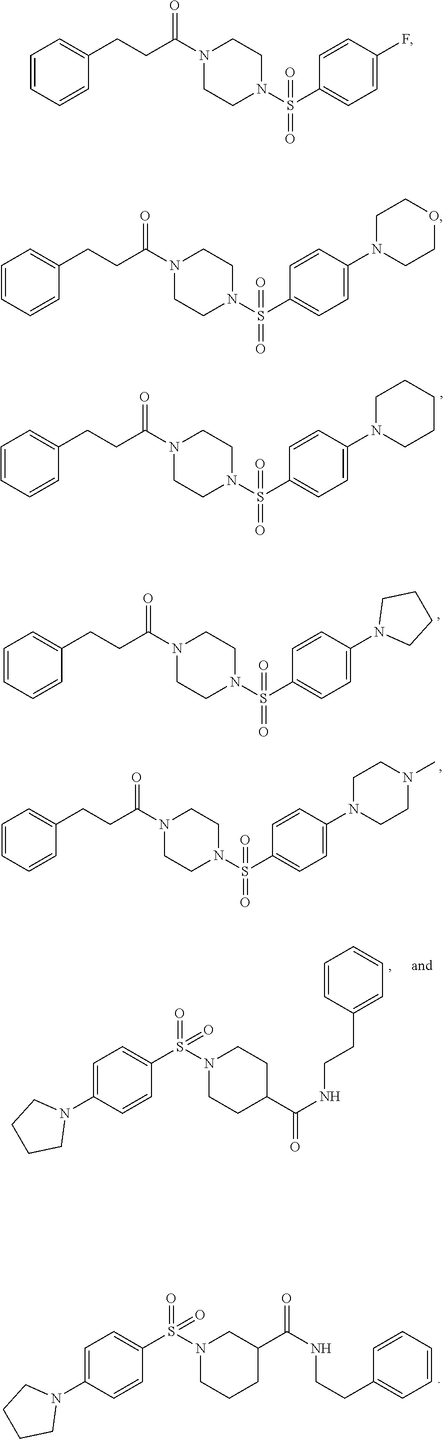

In another embodiment, R₁—X-L- in Formula I is

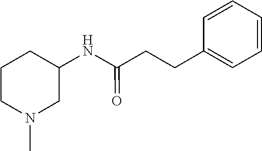 [SMILES: CN1CCCC(NC(=O)CCc2ccccc2)C1]

and the compound has the following Formula III:

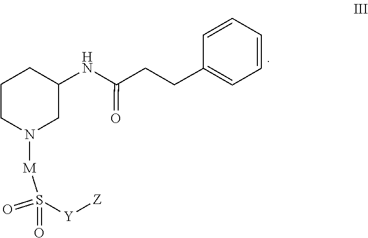

M is a bond, alky, aryl, a heterocyclic bivalent group, a heteroaromatic bivalent group, or an aromatic bivalent group; and Y is a bond, —NH—, a heterocyclic bivalent group, a heteroaromatic bivalent group, a bivalent benzyl group, or an aromatic bivalent group; Z is hydrogen, halogen, alkyl, aryl, heterocyclyl, heteroaryl, —NR₇R₈, —CO—R₁₀, or —NH—CO—R₁₀; R₇ and R₈ are independently hydrogen, hydroxy, halogen, nitro, amino, alkyl, alkoxy, alkylamino, cycloalkyl, cycloalkyamino, heterocyclyl, or heteroaryl; and R₁₀ is —O-tert-butyl, —CH₂CH₂-phenyl, hydrogen, hydroxy, halogen, nitro, amino, alkyl, alkoxy, alkylamino, cycloalkyl, cycloalkyamino, heterocyclyl, or heteroaryl.

In another embodiment, in Formula III, M is a bond; Y is a bivalent benzyl group; and Z is hydrogen or halogen.

In another embodiment, the compound is selected from the group consisting of

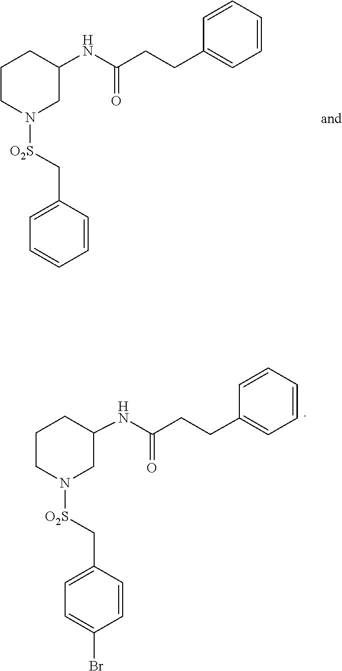 [SMILES: O=C(CCc1ccccc1)NC1CCCN(S(=O)(=O)Cc2ccccc2)C1 ; O=C(CCc1ccccc1)NC1CCCN(S(=O)(=O)Cc2ccc(Br)cc2)C1]

In another embodiment, in Formula III, M is a bivalent phenyl group; Y is a bond or —NH—; Z is hydrogen, heterocyclyl, heteroaryl, —NR₇R₈, —CO—R₁₀, or —NH—CO—R₁₀; R₇ and R₈ are independently hydrogen or alkyl; and R₁₀ is —O-tert-butyl, —CH₂CH₂-phenyl, hydrogen, hydroxy, halogen, nitro, amino, alkyl, alkoxy, alkylamino, cycloalkyl, cycloalkyamino, heterocyclyl, or heteroaryl.

In another embodiment, in Formula III, the compound is selected from the group consisting of:

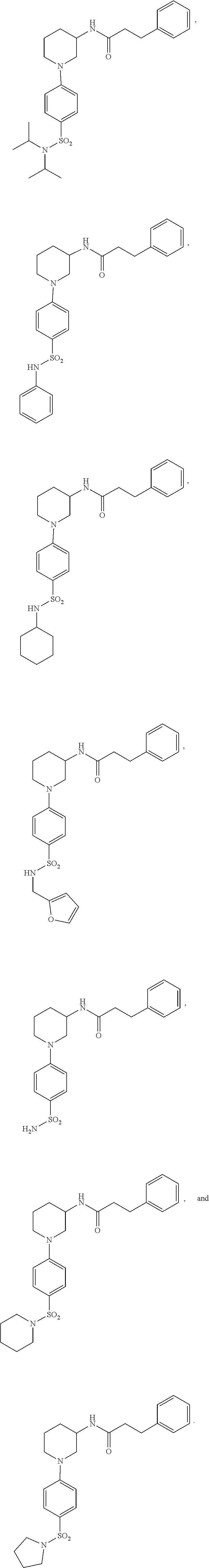

It is to be understood that both of the foregoing general description and the following detailed description are exemplary and explanatory and are intended to provide further explanation of the invention as claimed.

### DETAILED DESCRIPTION OF THE ILLUSTRATED EMBODIMENTS

Reference will now be made in detail to embodiments of the present invention.

The methods described herein include administering to a subject in need a composition containing a therapeutically effective amount of one or more purinergic receptor P2X7 (P2X7R) antagonists described herein, including enantiomerically pure forms thereof, and pharmaceutically acceptable salts or co-crystals and prodrugs thereof.

Prodrugs mean any compounds which release an active parent drug according to Formulas I-III in vivo when such prodrug is administered to a mammalian subject. Prodrugs of a compound are prepared by modifying functional groups present in the compounds of Formulas I-III in such a way that the modifications may be cleaved in vivo to release the parent compound. Prodrugs may be prepared by modifying functional groups present in the compounds in such a way that the modifications are cleaved, either in routine manipulation or in vivo, to the parent compounds.

Tautomers mean compounds produced by the phenomenon wherein a proton of one atom of a molecule shifts to another atom. Tautomers also refer to one of two or more structural isomers that exist in equilibrium and are readily converted from one isomeric form to another. One of ordinary skill in the art would recognize that other tautomeric ring atom arrangements are possible. All such isomeric forms of these compounds are expressly included in the present disclosure.

Isomers mean compounds having identical molecular formulas but differ in the nature or sequence of bonding of their atoms or in the arrangement of their atoms in space. Isomers that differ in the arrangement of their atoms in space are termed stereoisomers. Stereoisomers that are not mirror images of one another are termed diastereomers, and those that are non-superimposable mirror images of each other are termed enantiomers. When a compound has an asymmetric center, for example, it is bonded to four different groups, a pair of enantiomers is possible. A chiral compound can exist as either an individual enantiomer or as a mixture thereof. Unless otherwise indicated, the description is intended to include individual stereoisomers as well as mixtures.

Certain compounds of the present disclosure can exist in unsolvated forms as well as solvated forms, including hydrated forms. Solvates refer to a complex formed by combination of solvent molecules with the compound of Formulas I-III. The solvent can be an organic compound, an inorganic compound, or a mixture thereof.

The subject invention also includes isotopically-labeled compounds, which are identical to those listed in Formulas I-III and following, but one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, phosphorous, fluorine, iodine, and chlorine, such as ²H, ³H, ¹¹C, ¹⁴C, ¹⁸F, ¹²³I and ¹²⁵I. Compounds of Formulas I-III and pharmaceutically acceptable salts of said compounds that contain the aforementioned isotopes and/or other isotopes of other atoms are within the scope of the present invention. Isotopically-labeled compounds of the present invention are useful in medical diagnoses and therapeutic treatments. With different isotopes, such as H, ³H, ¹¹C, ¹⁴C, ¹⁸F, ¹²³I and ¹²⁵I, isotopically-labeled compounds of the present invention have their broad applications in medical diagnoses and therapeutic treatments. Isotopically labeled compounds of Formulas I-III, and following of this invention can generally be prepared by carrying out the procedures disclosed in the Scheme A-E and/or in the Examples below, by substituting a readily available isotopically labeled reagent for a non-isotopically labeled reagent.

Pharmaceutically acceptable salts represent those salts which are, within the scope of medical judgment, suitable for use in contact for the tissues of humans and lower animals without undue toxicity, irritation, allergic response and the like, and are commensurate with a reasonable benefit/risk ratio. They may be obtained during the final isolation and purification of the compounds of the invention, or separately by reacting the free base function with a suitable mineral acid such as hydrochloric acid, phosphoric acid, or sulfuric acid, or with an organic acid such as for example ascorbic acid, citric acid, tartaric acid, lactic acid, maleic acid, malonic acid, fumaric acid, glycolic acid, succinic acid, propionic acid, acetic acid, methanesulfonic acid, and the like. The acid function can be reacted with an organic or a mineral base, like sodium hydroxide, potassium hydroxide or lithium hydroxide.

Therapeutically effective amount means an amount of compound or a composition of the present invention effective to activate purinergic receptor P2X7 and to produce the desired therapeutic effect.

As used herein, the term alkyl refers to a monovalent straight or branched chain, saturated aliphatic hydrocarbon radical having a number of carbon atoms in the specified range. For example, C₁₋₆ alkyl refers to any of the hexyl alkyl and pentyl alkyl isomers as well as n-, iso-, sec- and t-butyl, n- and iso-propyl, ethyl and methyl. Alkyl also includes saturated aliphatic hydrocarbon radicals wherein one or more hydrogens are replaced with deuterium, for example, CD₃.

The term branched alkyl refers to an alkyl group as defined above except that straight chain alkyl groups in the specified range are excluded. As defined herein, branched alkyl includes alkyl groups in which the alkyl is attached to the rest of the compound via a secondary or tertiary carbon. For example, isopropyl is a branched alkyl group.

The term cycloalkyl refers to any monocyclic ring of an alkane having a number of carbon atoms in the specified range. For example, C₃₋₆cycloalkyl refers to cyclopropyl, cyclobutyl, cyclopentyl, and cyclohexyl.

The term halogen refers to fluorine, chlorine, bromine and iodine (alternatively referred to as fluoro, chloro, bromo, and iodo).

The term haloalkyl refers to an alkyl group as defined above in which one or more of the hydrogen atoms have been replaced with a halogen (i.e., F, Cl, Br and/or I). For example, C₁₋₆ haloalkyl refers to a C₁ to C₆ linear or branched alkyl group as defined above with one or more halogen substituents. The term fluoroalkyl has an analogous meaning except that the halogen substituents are restricted to fluoro. Suitable fluoroalkyls include the series (CH₂)₀₋₄CF₃.

The term C(O) or CO refers to carbonyl. The terms S(O)₂ or SO₂ refers to sulfonyl. The term S(O) or SO refers to sulfinyl.

The term aryl (aromatic group) refers to phenyl, naphthyl, tetrahydronaphthyl, idenyl, dihydroindenyl and the like. An aryl of particular interest is phenyl.

The term heteroaryl (heteroaromatic group) refers to (i) a 5- or 6-membered heteroaromatic ring containing from 1 to 4 heteroatoms independently selected from N, O and S, or (ii) is a heterobicyclic ring selected from quinolinyl, isoquinolinyl, and quinoxalinyl. Suitable 5- and 6-membered heteroaromatic rings include, for example, pyridyl (also referred to as pyridinyl), pyrrolyl, pyrazinyl, pyrimidinyl, pyridazinyl, triazinyl, thienyl, furanyl, imidazolyl, pyrazolyl, triazolyl, tetrazolyl, oxazolyl, isooxazolyl, oxadiazolyl, oxatriazolyl, thiazolyl, isothiazolyl, and thiadiazolyl. A class of heteroaryls of interest consists of (i) 5- and 6-membered heteroaromatic rings containing from 1 to 3 heteroatoms independently selected from N, O and S, and (ii) heterobicyclic rings selected from quinolinyl, isoquinolinyl, and quinoxalinyl. Heteroaryls of particular interest are pyrrolyl, imidazolyl, pyridyl, pyrazinyl, quinolinyl (or quinolyl), isoquinolinyl (or isoquinolyl), and quinoxalinyl.

Examples of 4- to 7-membered, saturated heterocyclic rings within the scope of this invention include, for example, azetidinyl, piperidinyl, morpholinyl, thiomorpholinyl, thiazolidinyl, isothiazolidinyl, oxazolidinyl, isoxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, azepanyl, diazepanyl, tetrahydropyranyl, tetrahydrothiopyranyl, and dioxanyl. Examples of 4- to 7-membered, unsaturated heterocyclic rings within the scope of this invention include mono-unsaturated heterocyclic rings corresponding to the saturated heterocyclic rings listed in the preceding sentence in which a single bond is replaced with a double bond (e.g., a carbon-carbon single bond is replaced with a carbon-carbon double bond).

It is understood that the specific rings listed above are not a limitation on the rings which can be used in the present invention. These rings are merely representative.

Synthetic methods for preparing the compounds of the present invention are illustrated in the following Schemes, Methods, and Examples. Starting materials are commercially available or may be prepared according to procedures known in the art or as described herein. The compounds of the invention are illustrated by means of the specific examples shown below. However, these specific examples are not to be construed as forming the only genus that is considered as the invention. These examples further illustrate details for the preparation of the compounds of the present invention. Those skilled in the art will readily appreciate that known variations in the conditions and processes can be used to prepare such compounds.

Compounds of present invention, and their salts and solvates thereof may be prepared by the methodology described hereinafter, constituting a further aspect of this invention.

Certain compounds of the present application may be prepared according to the following schemes and the knowledge of one skilled in the art. All temperatures are in degrees Celsius. Common abbreviations are applied.

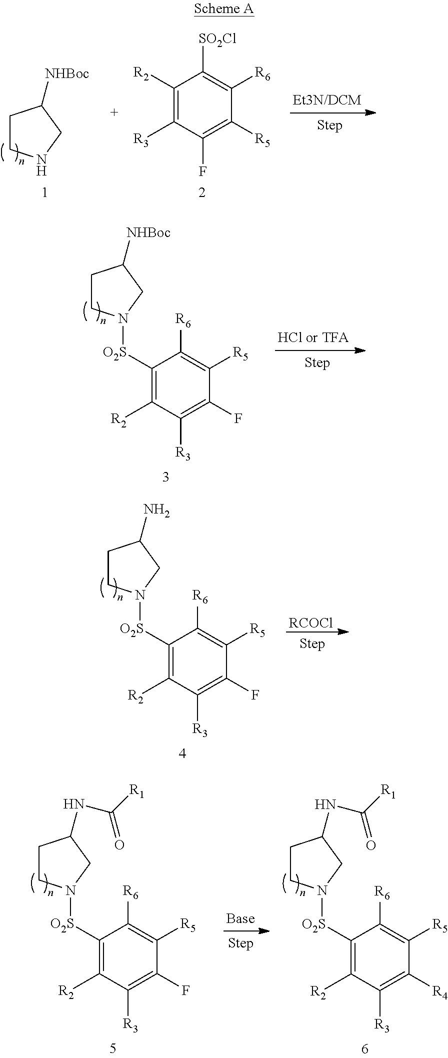

Step A1: The first step was accomplished by the reaction of compound (1) with sulfonyl chlorides (2) in different solvents and different temperatures using triethylamine or other bases to form sulfonamides (3). Compound (1) can be those amines listed as L in the Formulas I-III.

Step A2: The protecting groups of the amines in (3) was removed by the treatment with acids, such as trifluoroacetic acid or concentrated hydrochloric acid. In the case of Cbz protected hydrogenolysis was employed, it afforded free amines (4).

Step A3: The reaction of compounds (4) with all kind acyl chlorides using reasonable bases afforded amides (5).

Step A4: A replacement of fluorine atom of compound (5) using common reactions with amines, alcohols or thiols afforded compounds (6) of Scheme A.

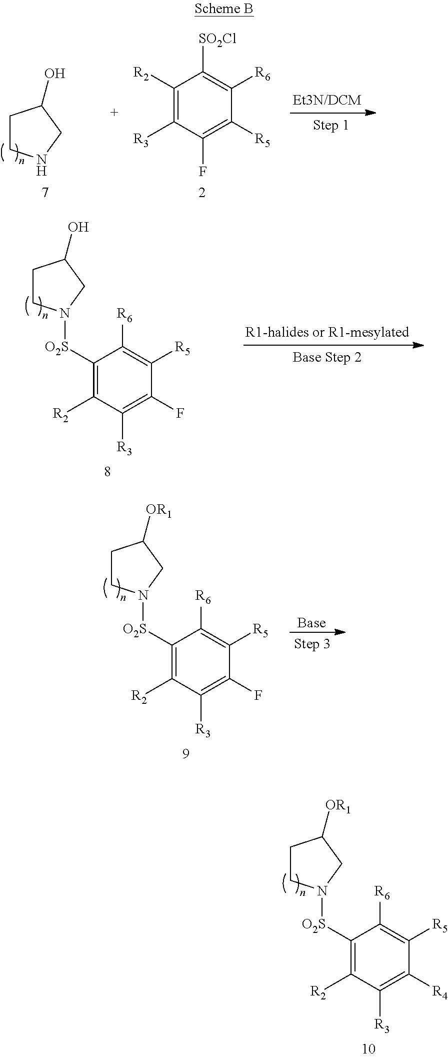

Step B1: Compound (7) of Scheme B was reacted with compound (2) using a similar reaction condition as described in Step A1 of Scheme A to provide sulfonamide (8).

Step B2: A reaction of the alcohol of compound (8) with alkyl halides or alkyl sulfates in the presence of an appropriate base, such as sodium hydride, afforded compound (9).

Step B3: A replacement of the fluorine atom of compound (9) in the Scheme B with amines, alcohols or thiols at the elevated temperatures in certain solvents, such DMSO, produced compounds (10).

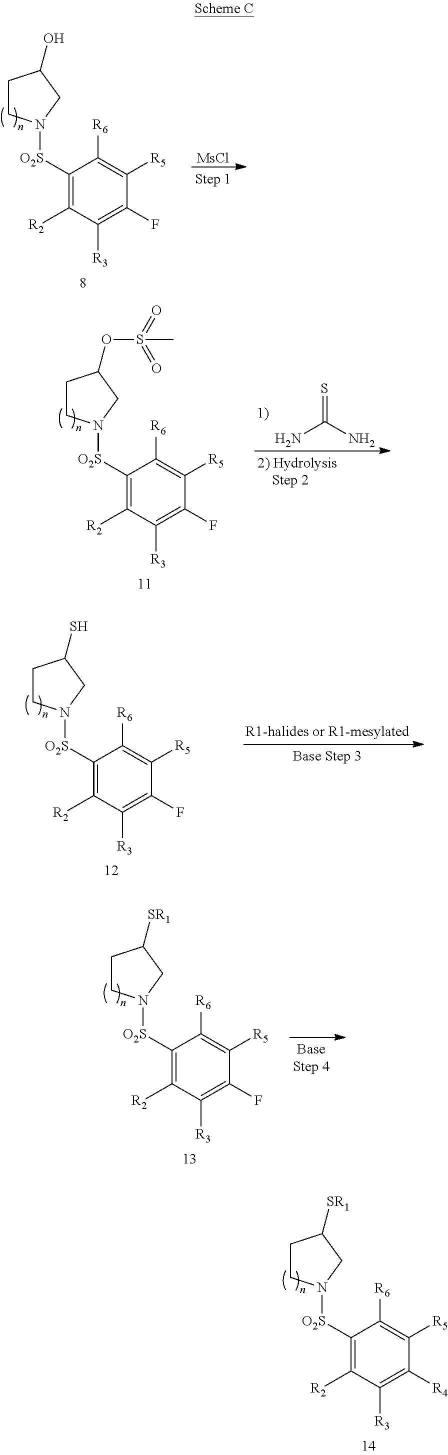

Step C1: Conversion of the alcohol of compound (8) in the Scheme C with methanesulfonyl chloride in the presence a common base afforded mesylate (11).

Step C2: Compound (12) was accomplished by a reaction of mesylate (11) with thiourea and then base hydrolysis.

Step C3: The alkylation of sulfur atom of compound (12) with alkyl halides or mesylates afforded compound (13).

Step C4: A replacement of fluorine atom in compound (13) using common reactions with amines, alcohols or thiols afforded compounds (14) of Scheme C.

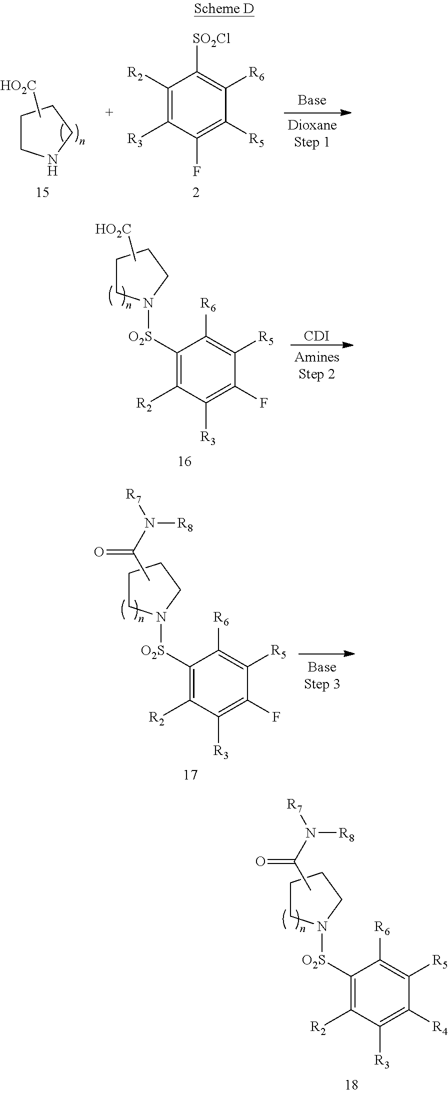

Step D1: Compound (15) (available in Combi-Blocks, San Diego, Calif.) was dissolved in aqueous sodium hydroxide and cooled to 0 degree, followed by adding sulfonyl chloride (2) in dioxane while stirring. The reaction was monitored by HPLC. The mixture was then neutralized using 2 N HCl to pH 7 after the reaction was over. The dioxane was removed and the solid was collected. The solid was washed with water and dried to afford compound (16) of Scheme D.

Step D2: Compound (16) was dissolved in dry DMF and carbonyl diimidazole (CDI) (Combi-Blocks) was added. The mixture was stirred for 1 to 2 hours and amines (Combi-Blocks) was added. The reaction was monitored by HPLC and poured into water once the reaction was done. The precipitates were collected and washed with 1 N aqueous HCl, aqueous sodium bicarbonate solution and water, and dried to afford compound (17) of Scheme D.

Step D3: Compound (17) was dissolved in DMSO and a base was added, followed by adding the R₄ building block. The mixture was stirred at an elevated temperature. The reaction was monitored by HPLC and cooled to room temperature once the reaction was completed. The mixture was poured into water to precipitate. The precipitates were collected and washed with water. The final compound (18) of Scheme D was further purified by recrystallization or column chromatograph. Adding other building blocks to the position of R₂, R₃, R₅, or R₆ may be achieved in a similar manner.

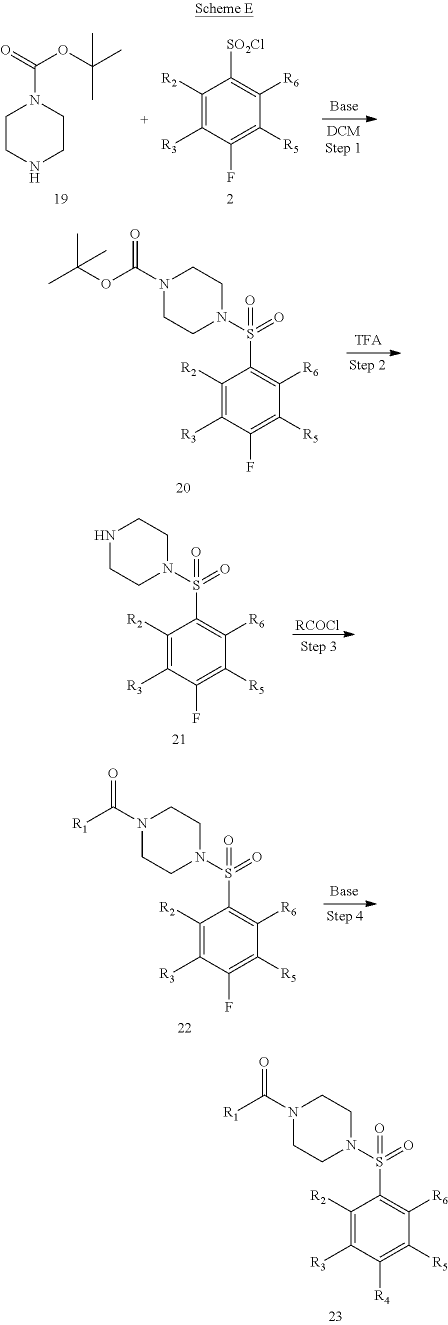

Step E1: Compound (19) (Combi-Blocks) was dissolved in DCM and cooled to 0 degree. Then triethylamine was added followed by adding sulfonyl chloride (2) in DCM dropwise. The reaction was monitored by HPLC. The product was washed by 1 N HCl aqueous solution and water. The organic layer was taken and dried over magnesium sulfate. The solution was concentrated to afford compound (20) in Scheme E.

Step E2: Compound (20) was added to TFA at room temperature and the resulting solution was stirred at 40 degree to remove the Boc protection group. The mixture was cooled to 0 degree once the reaction was over, and then made it alkaline by an aqueous base to afford compound (21).

Step E3: Compound (21) was dissolved in dioxane and cooled to 0 degree. After triethylamine was added, it followed by acyl chloride dropwise. The reaction was monitored by HPLC. The dioxane was removed and the residue was triturated with water to afford a solid compound (22) of Scheme E.

Step E4: Compound (22) was dissolved in DMSO and a base was added, followed by adding the R₄ building block. The mixture was stirred at an elevated temperature. The reaction was monitored by HPLC and cooled to room temperature once the reaction was completed. The solid was collected and purified by recrystallization or column chromatograph to afford a pure final compound (23) of Scheme E. Adding other building blocks to the position of R₂, R₃, R₅, or R₆ may be achieved in a similar manner.

The following examples illustrate the present invention prepared according to the above described schemes and the knowledge of one skilled in the art.

### Example 1

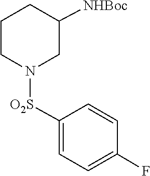 [SMILES: CC(C)(C)OC(=O)NC1CCCN(S(=O)(=O)c2ccc(F)cc2)C1]

To a solution of 3-Boc-aminopiperidine (100 mmol) in dichloromethane (100 mL) at 0 degree, triethylamine (110 mmol) was added, followed by adding 4-fluorobenzenesulfonyl chloride (100 mmol) in dichloromethane dropwise while stirring. The reaction was monitored by HPLC. Once the reaction was over, it was washed with 1 N HCl solution and NaHCO₃ solution sequentially. The dichloromethane solution was taken and concentrated to afford a solid Example 1.

### Example 2

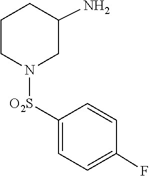 [SMILES: NC1CCCN(S(=O)(=O)c2ccc(F)cc2)C1]

Example 1 (100 mmol) was dissolved in dichloromethane (200 mL) and concentrated HCl (175 mL) was added dropwise while the reaction solution was stirred vigorously. Keep stirring at 40 degree after addition was completed until the HPLC analysis showed no more Example 1. The reaction solution was cooled to room temperature and the aqueous layer was taken. The aqueous layer was neutralized with 4N NaOH solution to pH 14. The white precipitates were collected, washed with water. and dried to afford Example 2.

### Example 3

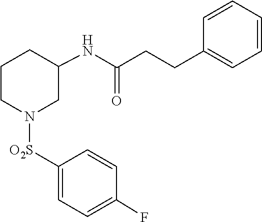 [SMILES: O=C(CCc1ccccc1)NC1CCCN(S(=O)(=O)c2ccc(F)cc2)C1]

Example 2 (50 mmol) was dissolved in dichloromethane (100 mL) and cooled to 0 degree and triethylamine (55 mmol) was added, followed by adding hydrocinnamoyl chloride (50 mmol) in dichloromethane (30 mL) dropwise. The reaction was monitored by HPLC. When the reaction was done, 1 N HCl solution (100 mL) was added. The organic layer was collected and washed with sodium bicarbonate solution. The solution was then dried over magnesium sulfate and concentrated, affording Example 3 as a white solid.

### Example 4

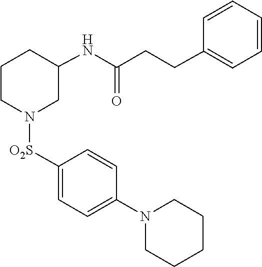 [SMILES: O=C(CCc1ccccc1)NC1CCCN(S(=O)(=O)c2ccc(N3CCCCC3)cc2)C1]

Example 3 (10 mmol) was dissolved in DMSO (50 mL) and potassium carbonate (20 mmol) was added, followed by adding piperidine (12 mmol). The mixture was stirred at 60 to 100 degree for 1 to 2 hours and then cooled down to room temperature. Water was added to precipitate. The precipitates were collected and washed by 1 N HCl. The solid was then dried and re-dissolved in ethyl acetate. The ethyl acetate solution was passed through a silica gel pad and concentrated to afford a pure white solid Example 4 in 80% yield.

### Example 5

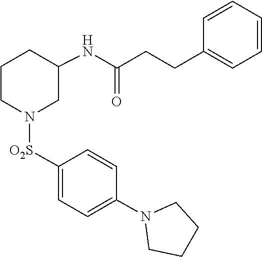 [SMILES: O=C(CCc1ccccc1)NC1CCCN(S(=O)(=O)c2ccc(N3CCCC3)cc2)C1]

Instead of piperidine as in Example 4, when pyrrolidine (12 mmol) was used, it afforded Example 5 as a white solid in 75% yield.

### Example 6

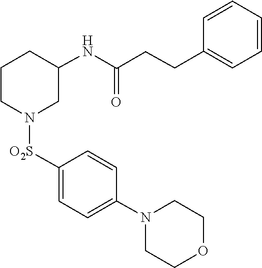 [SMILES: O=C(CCc1ccccc1)NC1CCCN(S(=O)(=O)c2ccc(N3CCOCC3)cc2)C1]

When morpholine was used instead of piperidine in Example 4, Example 6 was obtained as a white solid in 85% yield.

### Example 7

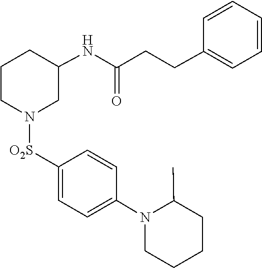 [SMILES: CC1CCCCN1c1ccc(S(=O)(=O)N2CCCC(NC(=O)CCc3ccccc3)C2)cc1]

When 2-methylpiperidine was used instead of piperidine in Example 4, Example 7 was afforded as a solid in 65% yield.

### Example 8

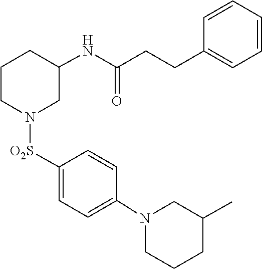 [SMILES: CC1CCCN(c2ccc(S(=O)(=O)N3CCCC(NC(=O)CCc4ccccc4)C3)cc2)C1]

When 3-methylpiperidine was employed instead of piperidine in Example 4, Example 8 was obtained in 80% yield.

### Example 9

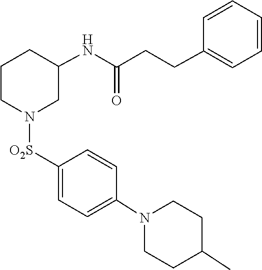 [SMILES: CC1CCN(c2ccc(S(=O)(=O)N3CCCC(NC(=O)CCc4ccccc4)C3)cc2)CC1]

When 4-methylpiperidine was used instead of piperidine in Example 4, Example 9 was obtained as a white solid in 78% yield.

### Example 10

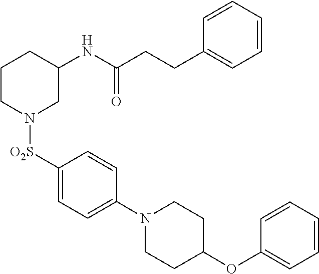 [SMILES: O=C(CCc1ccccc1)NC1CCCN(S(=O)(=O)c2ccc(N3CCC(Oc4ccccc4)CC3)cc2)C1]

When 4-phenoxypiperidine (Combi-Blocks) was used instead of piperidine in Example 4, Example 10 was obtained as a white solid in 77% yield.

### Example 11

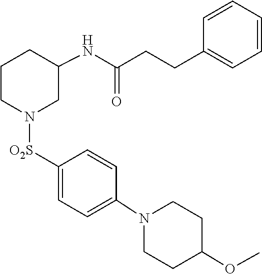 [SMILES: COC1CCN(c2ccc(S(=O)(=O)N3CCCC(NC(=O)CCc4ccccc4)C3)cc2)CC1]

When 4-methoxypiperidine was used to react with Example 3 using a similar reaction as in the Example 4, it afforded Example 11 as a white solid in 90% yield.

### Example 12

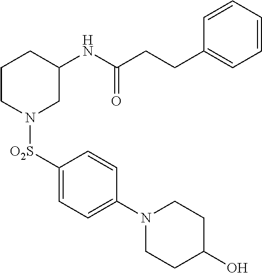 [SMILES: O=C(CCc1ccccc1)NC1CCCN(S(=O)(=O)c2ccc(N3CCC(O)CC3)cc2)C1]

When piperidine-4-ol was used instead of piperidine as in Example 4, it gave the crude products, which was purified by silica gel chromatograph to afford a pure Example 12 in 45% yield.

### Example 13

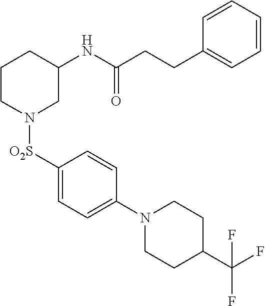 [SMILES: O=C(CCc1ccccc1)NC1CCCN(S(=O)(=O)c2ccc(N3CCC(C(F)(F)F)CC3)cc2)C1]

When 4-trifluoromethylpiperidine (Combi-Blocks) was used to react with Example 3 in a similar reaction as in Example 4, it produced Example 13 in 80% yield.

### Example 14

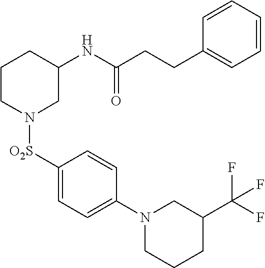 [SMILES: O=C(CCc1ccccc1)NC1CCCN(S(=O)(=O)c2ccc(N3CCCC(C(F)(F)F)C3)cc2)C1]

When the same reaction condition was employed as in Example 13, using 3-trifluoromethylpiperidine instead of 4-trifluoromethylpiperidine, it produced Example 14 in 70% yield.

### Example 15

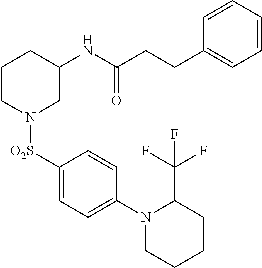 [SMILES: O=C(CCc1ccccc1)NC1CCCN(S(=O)(=O)c2ccc(N3CCCCC3C(F)(F)F)cc2)C1]

When 2-trifluoromethylpiperidine (Combi-Blocks) was used instead of piperidine as in Example 4 under sealed tube, it generated Example 15 in 30% yield.

### Example 16

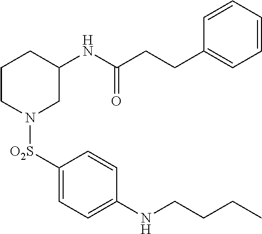 [SMILES: CCCCNc1ccc(S(=O)(=O)N2CCCC(NC(=O)CCc3ccccc3)C2)cc1]

When excess butylamine was used instead of piperidine in Example 4, it generated Example 16 in 78% yield.

### Example 17

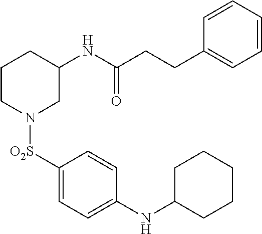 [SMILES: O=C(CCc1ccccc1)NC1CCCN(S(=O)(=O)c2ccc(NC3CCCCC3)cc2)C1]

When cyclohexylamine was used instead of piperidine as in Example 4, it afforded Example 17 in 80% yield.

### Example 18

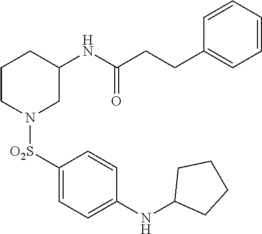 [SMILES: O=C(CCc1ccccc1)NC1CCCN(S(=O)(=O)c2ccc(NC3CCCC3)cc2)C1]

When cyclopentylamine was used instead of cyclohexylamine in a similar reaction condition as employed in Example 17, Example 18 was obtained.

### Example 19

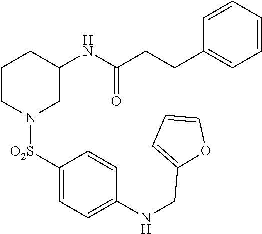 [SMILES: O=C(CCc1ccccc1)NC1CCCN(S(=O)(=O)c2ccc(NCc3ccco3)cc2)C1]

When furan-2-methylamine (Combi-Blocks) was used instead of piperidine as in Example 4, it afforded Example 19 in 65% yield.

### Example 20

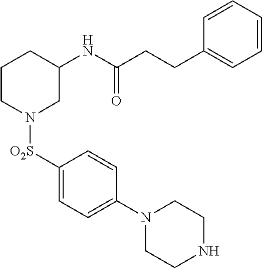 [SMILES: O=C(CCc1ccccc1)NC1CCCN(S(=O)(=O)c2ccc(N3CCNCC3)cc2)C1]

Step 20A: When t-butyl piperazine-1-carboxylate (Combi-Blocks) was used instead of piperidine as in Example 4, it produced Boc protected compound (Compound 20A) in 90% yield.

Step 20B: Boc protected material (100 mg) from Step 20A was dissolved in trifluoroacetic acid (5 mL) and stirred at room temperature for 2 hour. Then the TFA was removed and the residue was made alkaline using sodium hydroxide solution. The solid was collected and washed with water, and dried to afford Example 20 in 65% yield.

### Example 21

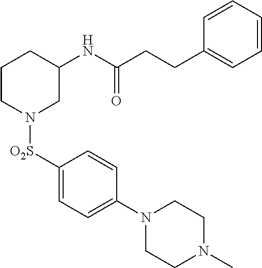 [SMILES: CN1CCN(c2ccc(S(=O)(=O)N3CCCC(NC(=O)CCc4ccccc4)C3)cc2)CC1]

When 1-methylpiperazine was employed instead of t-butyl piperidine-1-carboxylate as in Example 20, it directly produced Example 21 in 85% yield.

### Example 22

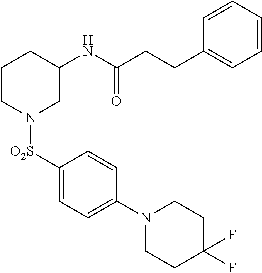 [SMILES: O=C(CCc1ccccc1)NC1CCCN(S(=O)(=O)c2ccc(N3CCC(F)(F)CC3)cc2)C1]

When 4,4-difluoropiperidine (Combi-Blocks) was used to replace the piperidine as in Example 4, it produced Example 22.

### Example 23

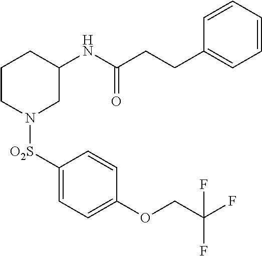 [SMILES: O=C(CCc1ccccc1)NC1CCCN(S(=O)(=O)c2ccc(OCC(F)(F)F)cc2)C1]

To a solution of Example 3 (10 mmol) in DMSO (50 mL), 2,2,2-trifluoroethanol (30 mmol) was added followed by adding sodium hydroxide (30 mmol). The mixture was stirred at 80 degree for 3 hours and then cooled to room temperature. Water (100 mL) was added to precipitate. The precipitates were collected and washed by 1 N HCl. The solid was then dried and re-dissolved in ethyl acetate. The ethyl acetate solution was passed through a silica gel pad and concentrated to afford a pure white solid of Example 23 in 80% yield.

### Example 24

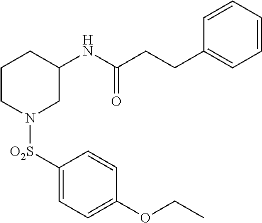 [SMILES: CCOc1ccc(S(=O)(=O)N2CCCC(NC(=O)CCc3ccccc3)C2)cc1]

When ethanol instead of 2,2,2-trfluoroethanol was used as in Example 23, Example 24 was obtained in 70% yield.

### Example 25

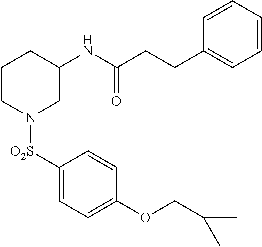 [SMILES: CC(C)COc1ccc(S(=O)(=O)N2CCCC(NC(=O)CCc3ccccc3)C2)cc1]

When isobutanol was used instead of ethanol as in Example 24, it afforded Example 25 in 60% yield.

### Example 26

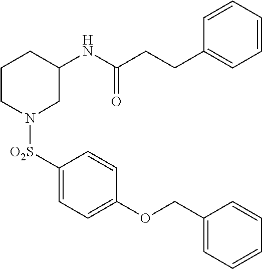 [SMILES: O=C(CCc1ccccc1)NC1CCCN(S(=O)(=O)c2ccc(OCc3ccccc3)cc2)C1]

To a solution of Example 3 (10 mmol) in DMSO (50 mL), benzyl alcohol (12 mmol) was added followed by adding sodium hydroxide (20 mmol). The mixture was stirred at 80 degree for 3 hours and then cooled to room temperature. Water (100 mL) was added to precipitate. The precipitates were collected and washed with water. The solid was then dried and re-dissolve in ethyl acetate. The ethyl acetate solution was passed through a silica gel pad and concentrated to afford a pure white solid of Example 26 in 85% yield.

### Example 27

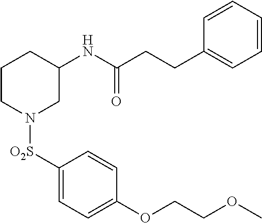 [SMILES: COCCOc1ccc(S(=O)(=O)N2CCCC(NC(=O)CCc3ccccc3)C2)cc1]

When 2-methoxyethanol was used instead of ethanol as in Example 24, it afforded Example 27 in 90% yield.

### Example 28

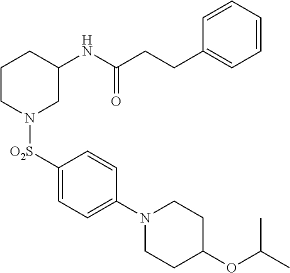 [SMILES: CC(C)OC1CCN(c2ccc(S(=O)(=O)N3CCCC(NC(=O)CCc4ccccc4)C3)cc2)CC1]

When 4-isopropoxypiperidine (Combi-Blocks) was used instead of piperidine as in Example 4, it generated Example 28 in 74% yield.

### Example 29

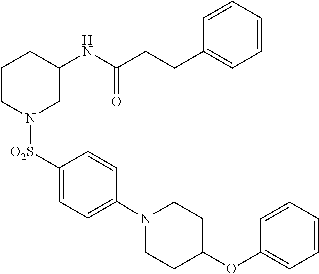 [SMILES: O=C(CCc1ccccc1)NC1CCCN(S(=O)(=O)c2ccc(N3CCC(Oc4ccccc4)CC3)cc2)C1]

When 4-phenoxypiperidine was used instead of piperidine as in Example 4, it produced Example 29.

### Example 30

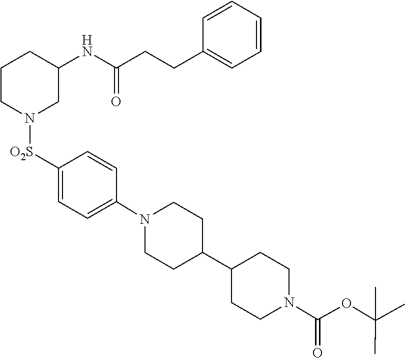 [SMILES: CC(C)(C)OC(=O)N1CCC(C2CCN(c3ccc(S(=O)(=O)N4CCCC(NC(=O)CCc5ccccc5)C4)cc3)CC2)CC1]

When N-boc-4,4′-bipiperidine (Combi-Blocks) was used instead of piperidine as in Example 4 and a similar procedure was employed, it produced Example 30 as a white solid in 78% yield.

### Example 31

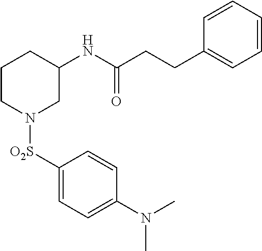 [SMILES: CN(C)c1ccc(S(=O)(=O)N2CCCC(NC(=O)CCc3ccccc3)C2)cc1]

To a solution of Example 3 (10 mmol) in DMF (20 mL), solid sodium hydroxide (20 mmol) was added. The mixture was stirred at 80 degree for 3 hours and then cooled to room temperature. Water (50 mL) was added to precipitate. The precipitates were collected and washed with water. The solid was then dried and re-dissolve in ethyl acetate. The ethyl acetate solution was passed through a silica gel pad and concentrated to afford a pure white solid of Example 31 in 70% yield.

### Example 32

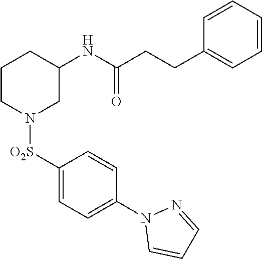 [SMILES: O=C(CCc1ccccc1)NC1CCCN(S(=O)(=O)c2ccc(-n3cccn3)cc2)C1]

Pyrazole (50 mmol) was mixed with Example 3 (10 mmol) in DMSO (50 mL) and potassium carbonate (20 mmol) was added. The resulting mixture was stirred at 120 degree for 3 hours. The reaction mixture was then cooled to room temperature and poured into water. The solid was collected and washed with water. The solid was purified by recrystallization using methanol to afford a pure material of Example 32 in 65% yield.

### Example 33

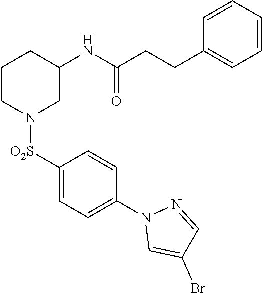 [SMILES: O=C(CCc1ccccc1)NC1CCCN(S(=O)(=O)c2ccc(-n3cc(Br)cn3)cc2)C1]

4-Bromopyrazole (Combi-Blocks) was employed to replace pyrazole using a similar reaction as in Example 32 to afford Example 33 in 70% yield.

### Example 34

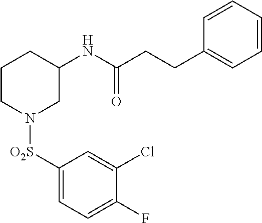 [SMILES: O=C(CCc1ccccc1)NC1CCCN(S(=O)(=O)c2ccc(F)c(Cl)c2)C1]

Step 34A: 1-N-Boc-piperidine-3-amine (Combi-Blocks) (100 mmol) was dissolved in DCM and cooled to 0 degree and triethylamine (110 mmol) was added, followed by adding hydrocinnamoyl chloride (100 mmol) dropwise while stirring. The reaction was monitored by HPLC. The mixture was washed with 1 N HCl aqueous solution, sodium bicarbonate solution, and water. The dichloromethane solution was taken and dried over magnesium sulfate. The DCM was removed to afford N-[3-(1-Boc-piperidyl)] hydrocinnamamido (Compound 34A) in a white solid in 95% yield.

Step 34B: Compound 34A (80 mmol) was dissolved in TFA and stirred at 40 degree. The reaction was monitored by HPLC. The mixture was made alkaline by adding aqueous sodium hydroxide solution to pH 14. The solid was collected, washed with water, and vacuum dried to afford N-(3-piperidyl) hydrocinnamate (Compound 34B) in gel.

Step 34C: Compound 34B (50 mmol) was dissolved in dioxane (100 mL) and triethylamine (60 mmol) was added. The mixture was cooled to 0 degree and 3-chloro-4-fluorobenzenesulfonyl chloride (Combi-Blocks) (50 mmol) was added in portions. The reaction was monitored by HPLC. The reaction mixture was concentrated by a rotavapor and the residue was triturated by water to afford a solid that was further purified by recrystallization in methanol to give Example 34 as a white solid in 90% yield.

### Example 35

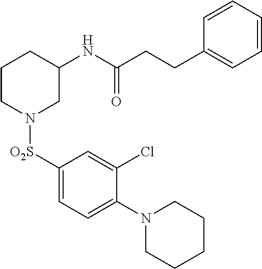 [SMILES: O=C(CCc1ccccc1)NC1CCCN(S(=O)(=O)c2ccc(N3CCCCC3)c(Cl)c2)C1]

Example 34 (1 mmol) in DMSO (15 mL) and piperidine (3 mmol) was added. The mixture was stirred at 100 degree for 2 hours and cooled to room temperature. Water was added and the precipitates were collected. The solid was purified by recrystallization in methanol to afford Example 35 in 78% yield.

### Example 36

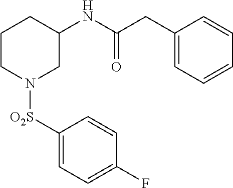 [SMILES: O=C(Cc1ccccc1)NC1CCCN(S(=O)(=O)c2ccc(F)cc2)C1]

Example 2 (50 mmol) was dissolved in DCM (150 mL) and triethylamine (55 mmol) was added. When the mixture was cooled to 0 degree, phenylacetyl chloride (50 mmol) was added dropwise. The reaction was monitored by HPLC. The reaction mixture was washed with 1 N HCl aqueous solution followed by aqueous sodium bicarbonate solution, and dried over magnesium sulfate. The solution was concentrated and the residue was purified by recrystallization in methanol to afford Example 36.

### Example 37

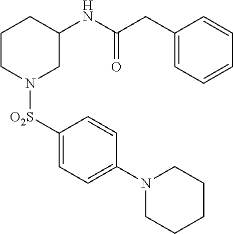 [SMILES: O=C(Cc1ccccc1)NC1CCCN(S(=O)(=O)c2ccc(N3CCCCC3)cc2)C1]

Example 36 (1 mmol) was dissolved in DMSO (15 mL) and piperidine (3 mmol) was added. The resulting mixture was then stirred at 100 degree for 2 hours. The mixture was cooled and poured into water. The precipitates were collected and purified by recrystallization in methanol to afford Example 37 in 85% yield.

### Example 38

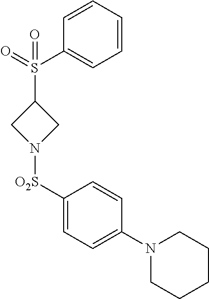 [SMILES: O=S(=O)(c1ccccc1)C1CN(S(=O)(=O)c2ccc(N3CCCCC3)cc2)C1]

Step 38A: 3-Phenylsulfonylazetidine (Combi-Blocks) (1 mmol) was dissolved in DMF (10 mL) and cooled to 0 degree. Then triethylamine (1.2 mmol) was added followed by adding 4-fluorobenzenesulfonyl chloride (1 mmol). The reaction was monitored by HPLC. Aqueous 1 N HCl solution was added and the organic layer was taken. The organic solution was dried over magnesium sulfate and concentrated to afford N-(4-fluorobenzenesulfonyl)-3-phenylsulfonylazetidine (Compound 38A) of Example 38.

Step 38B: Compound 38A of Step 38A (0.1 mmol) was dissolved in DMSO (5 mL) and piperidine (0.5 mmol) was added. The mixture was then stirred at 80 degree for 2 hours and cooled to room temperature. The mixture was poured into water and the solid was collected and washed with water. The solid was dried and purified by column chromatograph to afford Example 38 in a white solid in 65% yield.

### Example 39

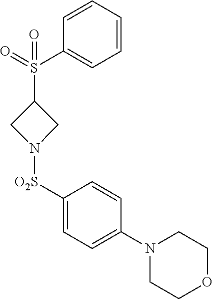 [SMILES: O=S(=O)(c1ccccc1)C1CN(S(=O)(=O)c2ccc(N3CCOCC3)cc2)C1]

Compound 38A in Example 38 (0.1 mmol) was dissolved in DMSO (5 mL) and morpholine (0.5 mmol) was added. The mixture was stirred at 100 degree for 3 hours. The reaction mixture was then cooled to room temperature and water was added. The solid was collected and purified by column chromatograph to afford Example 39.

### Example 40

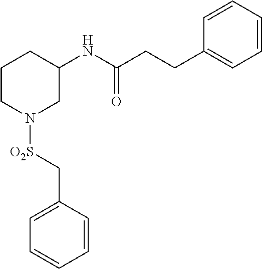 [SMILES: O=C(CCc1ccccc1)NC1CCCN(S(=O)(=O)Cc2ccccc2)C1]

Compound 34B, (3-piperidyl) hydrocinnamide, (0.1 mmol) in Example 34 was dissolved in DCM and triethylamine (0.11 mmol) was added. The mixture was cooled to 0 degree and benzylsulfonyl chloride (Combi-Blocks) (0.1 mmol) was added. The regular work up and purification by recrystallization in methanol afforded Example 40 in 95% yield.

### Example 41

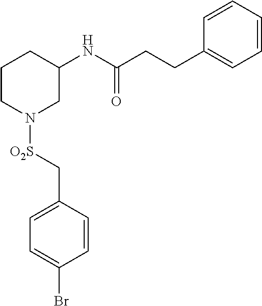 [SMILES: O=C(CCc1ccccc1)NC1CCCN(S(=O)(=O)Cc2ccc(Br)cc2)C1]

Instead of benzylsulfonyl chloride in Example 40, if 4-bromobenzylsulfonyl chloride (Combi-Blocks) was used in a same scale, it afforded Example 41.

### Example 42

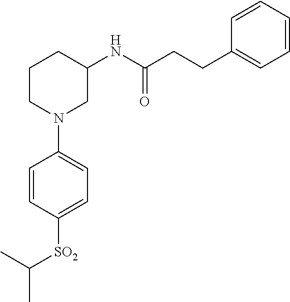 [SMILES: CC(C)S(=O)(=O)c1ccc(N2CCCC(NC(=O)CCc3ccccc3)C2)cc1]

Step 42A: Sodium 4-Fluorobezenesulfinate (Combi-Blocks) (1 mmol) was mixed in DMF (15 mL) and 2-iodopropane (5 mmol) was added. The mixture was then stirred at 60 degree under argon for 5 hours. The mixture was cooled down and water was added, and then was extracted with DCM. The DCM extract was washed with water and dried over magnesium sulfate. The DCM was removed to afford crude 1-fluoro-4-isopropylsulfonylbenzene (Compound 42A) that was pure enough for the next step.

Step 42B: Crude 1-fluoro-4-isopropylsulfonylbenzene (0.1 mmol) was dissolved in DMSO (15 mL) and Compound 34B, (3-piperidyl) hydrocinnamide, (0.1 mmol) in Example 34 was added, followed by adding potassium carbonate (0.5 mmol). The reaction mixture was stirred at 100 degree for 3 hours. The mixture was cooled to room temperature and water was added. The solid was collected and further purified by recrystallization in methanol to afford Example 42 in 78% yield.

### Example 43

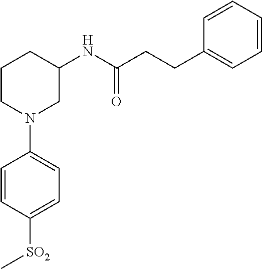 [SMILES: CS(=O)(=O)c1ccc(N2CCCC(NC(=O)CCc3ccccc3)C2)cc1]

When 1-fluoro-4-methylsulfonylbenzene (Combi-Blocks) was used instead of 1-fluoro-4-isopropylsulfonylbenzene in Example 42 using a similar procedure as in Example 42, it afforded Example 43.

### Example 44

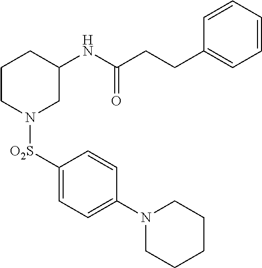 [SMILES: O=C(CCc1ccccc1)NC1CCCN(S(=O)(=O)c2ccc(N3CCCCC3)cc2)C1]

To a suspension of lithium alumina hydride (0.1 mmol) in THE (20 mL), a solution of Example 4 (0.1 mmol) in THE (20 mmmol) was added at room temperature. The resulting mixture was refluxed for 1 hour and cooled down to room temperature. The reaction mixture was quenched by adding aqueous sodium hydroxide solution. The resulting mixture was filtered and the THF solution was concentrated. The residue was purified by column chromatograph to afford Example 44.

### Example 45

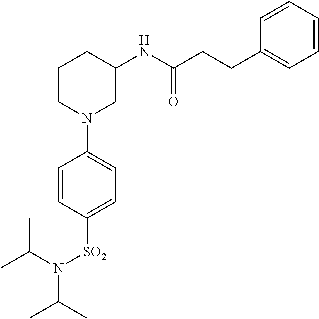 [SMILES: CC(C)N(C(C)C)S(=O)(=O)c1ccc(N2CCCC(NC(=O)CCc3ccccc3)C2)cc1]

Step 45A: To a solution of diisopropylamine (2 mmol) in DCM at 0 degree, 4-fluorobenzenesulfonyl chloride (1 mmol) was added. The reaction mixture was then washed with 1 N HCl aqueous solution. The DCM was then concentrated to afford N,N-diisopropyl 4-fluorobenzenesulfonamide (compound 45A) as a white solid.

Step 45B: A mixture of N,N-diisopropyl 4-fluorobenzenesulfonamide (0.1 mmol) and Compound 34B, (3-piperidyl) hydrocinnamide, (0.1 mmol) in Example 34 was stirred in DMSO in the presence of potassium carbonate (0.2 mmol) at 100 degree. The reaction was monitored by HPLC. The mixture was cooled and poured into water. The solid was collected, washed with water, and further purified by recrystallization in methanol to afford Example 45 as a white solid.

### Example 46

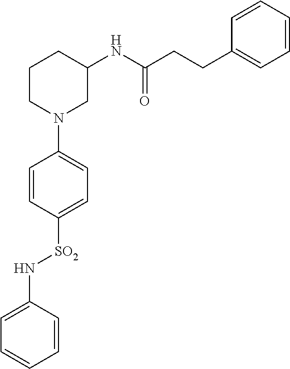 [SMILES: O=C(CCc1ccccc1)NC1CCCN(c2ccc(S(=O)(=O)Nc3ccccc3)cc2)C1]

When aniline was used instead of diisoproplyamine as in Example 45, a similar procedure as employed in Example 45 afforded Example 46.

### Example 47

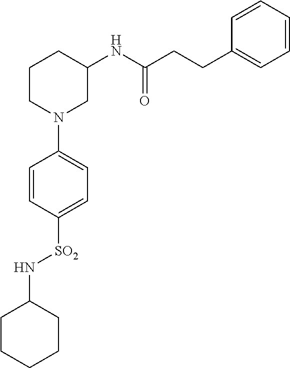 [SMILES: O=C(CCc1ccccc1)NC1CCCN(c2ccc(S(=O)(=O)NC3CCCCC3)cc2)C1]

To produce Example 47, cyclohexylamine was used instead of diisopropylamine and a similar procedure was employed as in Example 45.

### Example 48

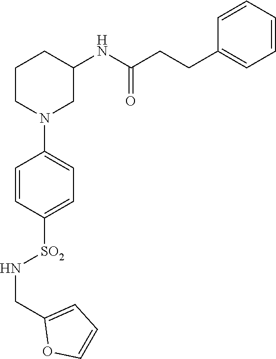 [SMILES: O=C(CCc1ccccc1)NC1CCCN(c2ccc(S(=O)(=O)NCc3ccco3)cc2)C1]

To produce Example 48, furan-2-methylamine was used instead of diisopropylamine and a similar procedure was employed as in Example 45.

### Example 49

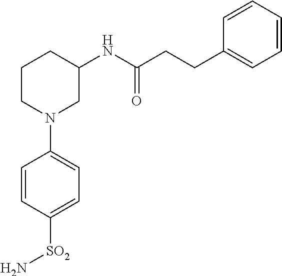 [SMILES: NS(=O)(=O)c1ccc(N2CCCC(NC(=O)CCc3ccccc3)C2)cc1]

Example 49 was also made in 40% yield using a similar procedure as in Example 45 when ammonia was used instead of diisopropylamine.

### Example 50

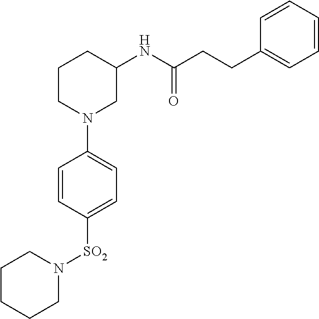 [SMILES: O=C(CCc1ccccc1)NC1CCCN(c2ccc(S(=O)(=O)N3CCCCC3)cc2)C1]

To afford Example 50 in 80% yield, piperidine was used instead of diisopropylamine as in Example 45 and also a similar procedure was employed as in Example 45.

### Example 51

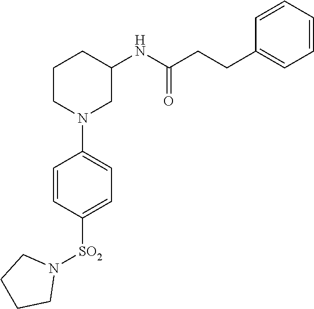 [SMILES: O=C(CCc1ccccc1)NC1CCCN(c2ccc(S(=O)(=O)N3CCCC3)cc2)C1]

To afford Example 51 in 80%, pyrrolidine was used instead of piperidine as in Example 50 and a similar procedure was employed

### Example 52

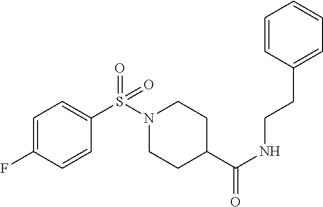 [SMILES: O=C(NCCc1ccccc1)C1CCN(S(=O)(=O)c2ccc(F)cc2)CC1]

Step 52A: Piperidine-4-carboxylic acid (100 mmol) was dissolved in aqueous 2 N sodium hydroxide solution (200 mmol) and dioxane (100 mL) was added. The mixture was cooled to 0 degree and 4-fluorobenzenesulfonyl chloride (100 mmol) was added in portions. The mixture was stirred at 0 degree until all the sulfonyl chloride disappeared. The mixture was then acidified with 4 N HCl aqueous solution. The solid was collected, washed with water, and dried to afford N-(4-Fluorobenzenesulfonyl) piperidine-4-carboxylic acid (Compound 52A) of Example 52.

Step 52B: Compound 52A (50 mmol) was dissolved in DMF and CDI (55 mmol) was added at 0 degree. The mixture was stirred for 1 hour and phenylethylamine (50 mmol) was added. The reaction was monitored by HPLC. After reaction, water was added and the precipitates were collected and washed with water. The solid was dried to afford Example 52 in 92% yield.

### Example 53

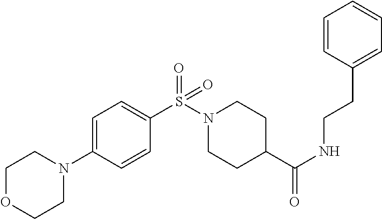 [SMILES: O=C(NCCc1ccccc1)C1CCN(S(=O)(=O)c2ccc(N3CCOCC3)cc2)CC1]

Example 52 (0.1 mmol) was dissolved in DMSO (10 mL) and morpholine (0.3 mmol) was added. The mixture was stirred at 80 degree for 2 hours. The mixture was cooled to room temperature and water was added. The solid was collected and purified by recrystallization in methanol to afford Example 53 in 85% yield.

### Example 54

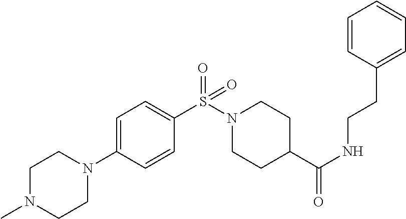 [SMILES: CN1CCN(c2ccc(S(=O)(=O)N3CCC(C(=O)NCCc4ccccc4)CC3)cc2)CC1]

To afford Example 54 in 82% yield, 1-methylpiperazine was used instead of morpholine as in Example 53 and a similar procedure was employed as in Example 53.

### Example 55

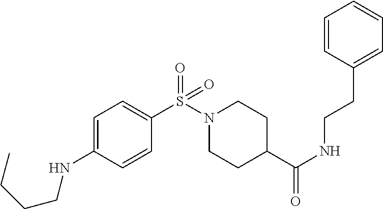 [SMILES: CCCCNc1ccc(S(=O)(=O)N2CCC(C(=O)NCCc3ccccc3)CC2)cc1]

To afford Example 55 in 55% yield, butylamine was used instead of morpholine and a similar procedure was employed as in Example 53.

### Example 56

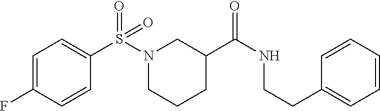 [SMILES: O=C(NCCc1ccccc1)C1CCCN(S(=O)(=O)c2ccc(F)cc2)C1]

Piperidine-3-carboxylic acid (100 mmol) was used instead of piperidine-4-carboxylic acid as in Example 52 and a similar procedure was also employed as in Example 52 to afford Example 56 as a white solid in 83% yield.

### Example 57

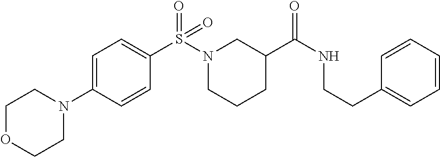 [SMILES: O=C(NCCc1ccccc1)C1CCCN(S(=O)(=O)c2ccc(N3CCOCC3)cc2)C1]

Using Example 56 (0.1 mmol) and employing a similar procedure of Example 53, it afforded Example 57 in 78% as a white solid.

### Example 58

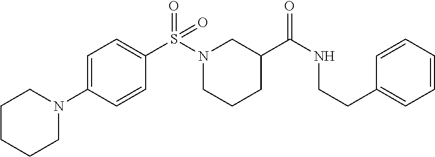 [SMILES: O=C(NCCc1ccccc1)C1CCCN(S(=O)(=O)c2ccc(N3CCCCC3)cc2)C1]

Piperidine was used instead of morpholine as in Example 57 to generate Example 58 in 81% yield.

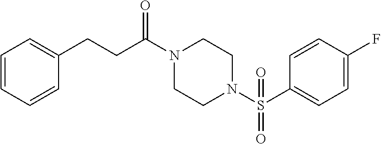 [SMILES: O=C(CCc1ccccc1)N1CCN(S(=O)(=O)c2ccc(F)cc2)CC1]

### Example 59

Step 59A: Compound 19 (100 mmol) was dissolved in DCM (200 mL) and triethylamine (110 mmol) was added. The mixture was cooled to 0 degree and hydrocinnamoyl chloride (100 mmol) was added dropwise. The reaction mixture was stirred at 0 degree for 1 hour and washed with 1 N HCl aqueous solution and sodium bicarbonate solution. The DCM solution was dried over magnesium sulfate and concentrated to afford Compound 59A in 95% yield.

Step 59B: Compound 59A (90 mmol) was dissolve in trifluoroacetic acid (50 mL) and stirred at 40 degree for 2 hours and trifluoroacetic acid was removed. The residue was made alkaline to pH 14 with 2 N sodium hydroxide at 0 degree and extracted with DCM. The DCM extract was dried and concentrated to afford Compound 59B in 75% yield.

Step 59C: Compound 59B (50 mmol) was dissolve in DCM (100 mL) and triethylamine (55 mmol) was added. The mixture was cooled to 0 degree and 4-fluorobenzenesulfonyl chloride (50 mmol) was added in portions. The mixture was then stirred at 0 degree for 2 hours and regular work up procedures were employed to afford Example 59 in 90% yield.

### Example 60

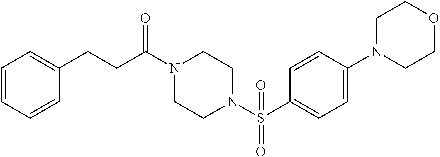 [SMILES: O=C(CCc1ccccc1)N1CCN(S(=O)(=O)c2ccc(N3CCOCC3)cc2)CC1]

Example 59 (0.1 mmol) was dissolved in DMSO (10 mL) and morpholine (0.3 mmol) was added. The reaction was stirred at 100 degree for 2 hours. The similar work up procedure was employed as in Example 53 to afford Example 60 in 80% yield.

### Example 61

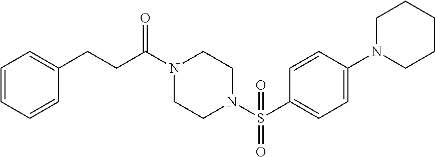 [SMILES: O=C(CCc1ccccc1)N1CCN(S(=O)(=O)c2ccc(N3CCCCC3)cc2)CC1]

Example 61 was obtained in 78% yield using a similar procedure as in Example 60 when piperidine was used instead of morpholine.

### Example 62

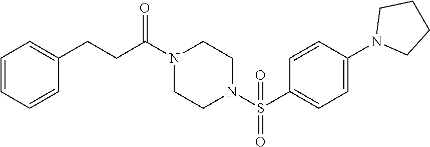 [SMILES: O=C(CCc1ccccc1)N1CCN(S(=O)(=O)c2ccc(N3CCCC3)cc2)CC1]

Example 62 was also obtained in 75% yield using a similar procedure as in Example 60 when pyrrolidine was used instead of morpholine.

### Example 63

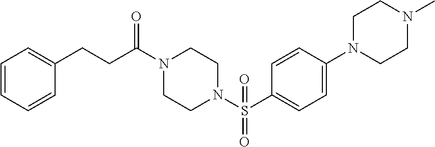 [SMILES: CN1CCN(c2ccc(S(=O)(=O)N3CCN(C(=O)CCc4ccccc4)CC3)cc2)CC1]

Example 63 was obtained in 86% yield as a white solid, using a similar procedure as in Example 60 when 1-methylpiperazine was used instead of morpholine.

### Example 64

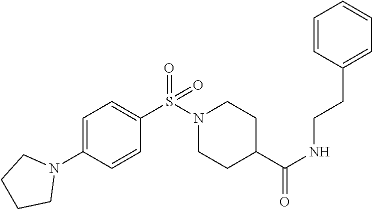 [SMILES: O=C(NCCc1ccccc1)C1CCN(S(=O)(=O)c2ccc(N3CCCC3)cc2)CC1]

Example 64 was obtained in 85% yield when pyrrolidine was used instead of morpholine using a similar procedure as in Example 53.

### Example 65

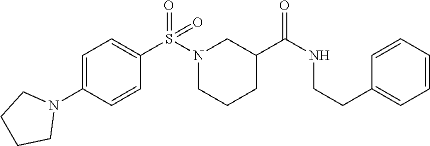 [SMILES: O=C(NCCc1ccccc1)C1CCCN(S(=O)(=O)c2ccc(N3CCCC3)cc2)C1]

When pyrrolidine was used instead of morpholine and a similar procedure was employed as in Example 57, it afforded Example 64 in 80% yield.

### PHARMACOLOGICAL EXAMPLES

Examples of the invention underwent biological studies are listed in Table 1. Biological assays used to characterize these compounds are described below.

### Examples of the Invention were Found to be P2X7R Antagonists Using an Assay Based on Yo-Pro-1 Uptake (Yo-Pro-1 Assay)

Yo-Pro-1 Iodide (Cat. No Y3603 from Fisher Scientific) is a fluorescent DNA-binding dye with a MW of 629 Da. This method is based upon the Yo-Pro-1 entrance to cells through the dilated or “large pore form” of P2X7R and its binding to intracellular DNA whereby it increases its fluorescence intensity (J. Pharmacol. Exp. Ther. 308, 1053-1061). The dye has an absorption spectrum compatible with excitation at 488 nm by argon laser sources and its emission wavelength is in the range of 515-575 nm. This Yo-Pro-1 uptake was measured in HEK-293 cells transiently transfected with P2X7R (J. Biol. Chem. 290, 7930-7942). In Brief, HEK-293 cells were seeded in growth medium at approximately 20,000 cells/well in a 96-well plate on day 1. Transfection was performed on day 2 using Lipofectamine 2000 with the P2X7R DNA plasmid at the concentration of 1.7 μg/ml. The transfection solution was replaced with the growth medium 4 hours later. Fluorescent assays were conducted on day 4. On day 4, the medium was removed, the cells were washed with phosphate buffered saline (PBS) (Cat. No. MT21040CV from Fisher Scientific). Then, 25 μl of PBS were first loaded to each well followed by 25 μl of various test compounds. The third 25 μl of solution contained a mixture of Yo-Pro-1 and stimulus bzATP (2′(3′)-O-(4-Benzoylbenzoyl)adenosine-5′-triphosphate tri(triethylammonium) salt). The final concentration of Yo-Pro-1 was 2.5 μM and of bzATP 30 μM. Fluorescence increases were measured every 30 minutes in a microplate device with excitation at 475 nm and emission wavelength in the range of 500-550 nm. PPADS (Pyridoxalphosphate-6-azophenyl-2′,4′-disulfonic acid tetrasodium) was used as the positive control. The wells with no test compounds added nor bzATP and only containing PBS and Yo-Pro-1 were served as the negative control (N Contl). The positive control (P Contl) wells were the same as N Contl but contained the stimulus, bzATP in this case.

Data used for analysis were from microplate readings 1 hour after bzATP stimulation. The result of each well was first normalized by its corresponding base microplate reading. The base microplate reading was the first reading right after all components were added. Then, the negative control signal was subtracted from. In the negative control, the test compound and the stimulus were replaced by PBS. An average of six highest values of the plate (AVG) were considered as 100% and the value of each well was divided by AVG and presented as the relative values in percentages.

With 30 μM bzATP as the stimulus, the half maximal inhibitory concentration (IC₅₀) of the antagonists from compounds of Formulas I-III was estimated based on dose-response curves spanning several logarithmic units and generated by the Prism software. The results are listed in Table 1.

For comparison of the antagonistic effectiveness of the compounds of this invention among P2X receptors, assays were conducted at 5 μM. Among 55 compounds tested, 29 can reduce the P2X7R activity to 1500 or less of the normal activity (Table 2).

TABLE 1
Compound Names, ID and Estimated IC₅₀

| Com- | | |
|---|---|---|
| pound | | IC₅₀ |
| ID | Chemical Name | (μM) |
| #1 | N-[1-(4-methanesulfonylphenyl)piperidin-3-yl]-3- | 3.9 |
| | phenylpropanamide | |
| #2 | N-{1-[(4-methoxybenzene)sulfonyl]piperidin-3-yl}-3- | 2.8 |
| | phenylpropanamide | |
| #3 | N-{1-[(4-Fluorobenzene)sulfonyl]piperidin-3-yl}-3- | 3.2 |
| | phenylpropanamide | |
| #4 | N-(1-{[4-(benzyloxy)benzene]sulfonyl}piperidin-3-yl)-3- | 8.2 |
| | phenylpropanamide | |
| #5 | 3-phenyl-N-(1-{[4-(piperidin-1- | 0.2 |
| | yl)benzene]sulfonyl}piperidin-3-yl)propanamide | |
| #6 | N-(1-{[4-(2-methylpropoxy)benzene]sulfonyl}piperidin- | 2.7 |
| | 3-yl)-3-phenylpropanamide | |
| #7 | 3-phenyl-N-{1-[4-(propane-2-sulfonyl)phenyl]piperidin-3- | 6.0 |
| | yl}propanamide | |
| #8 | N-(1-{[(4-bromophenyl)methane]sulfonyl}piperidin- | 2.1 |
| | 3-yl)-3-phenylpropanamide | |
| #9 | N-(1-{[4-(4-bromopyrazol-1- | 16.0 |
| | yl)benzene]sulfonyl}piperidin-3-yl)-3-phenylpropanamide | |
| #10 | 1-{4-[3-(benzenesulfonyl)azetidine-1- | 5.9 |
| | sulfonyl]phenyl}piperidine | |
| #11 | 4-{4-[3-(benzenesulfonyl)azetidine-1- | 9.9 |
| | sulfonyl]phenyl}morpholine | |
| #12 | N-(1-{[4-(morpholin-4-yl)benzene]sulfonyl}piperidin- | 6.7 |
| | 3-yl)-3-phenylpropanamide | |
| #13 | 3-phenyl-N-(1-{[4-(pyrrolidin-1- | 1.6 |
| | yl)benzene]sulfonyl}piperidin-3-yl)propanamide | |
| #14 | N-(1-{[4-(dimethylamino)benzene]sulfonyl}piperidin- | 1.4 |
| | 3-yl)-3-phenylpropanamide | |
| #15 | N-(1-{[4-(butylamino)benzene]sulfonyl}piperidin-3-yl)- | 1.0 |
| | 3-phenylpropanamide | |
| #16 | N-[2-(2,6-dioxopiperidin-3-yl)-1-oxo-3H-isoindol-4-yl]- | 3.6 |
| | 3-phenylpropanamide | |
| #17 | N-(1-{[4-(2-methoxyethoxy)benzene]sulfonyl}piperidin- | 16.1 |
| | 3-yl)-3-phenylpropanamide | |
| #18 | N-(1-{[4-(4-methylpiperazin-1- | 31.2 |
| | yl)benzene]sulfonyl}piperidin-3-yl)-3-phenylpropanamide | |
| #19 | N-(2-phenylethyl)-1-{[4-(piperidin-1- | 0.4 |
| | yl)benzene]sulfonyl}piperidine-3-carboxamide | |
| #20 | N-(2-phenylethyl)-1-{[4-(piperidin-1- | 1.0 |
| | yl)benzene]sulfonyl}piperidine-4-carboxamide | |
| #21 | N-(2-phenylethyl)-1-{[4-(pyrrolidin-1- | 0.9 |
| | yl)benzene]sulfonyl}piperidine-4-carboxamide | |
| #22 | tert-butyl 4-(4-{4-[(2-phenylethyl)carbamoyl]piperidine- | 12.6 |
| | 1-sulfonyl}phenyl)piperazine-1-carboxylate | |
| #23 | 1-{[4-(morpholin-4-yl)benzene]sulfonyl}-N-(2- | 26.6 |
| | phenylethyl)piperidine-4-carboxamide | |
| #24 | 1-{[4-(morpholin-4-yl)benzene]sulfonyl}-N-(2- | 10.2 |
| | phenylethyl)piperidine-3-carboxamide | |
| #25 | N-(2-phenylethyl)-1-{[4-(pyrrolidin-1- | 5.1 |
| | yl)benzene]sulfonyl}piperidine-3-carboxamide | |
| #26 | N-(2-phenylethyl)-1-{[4-(piperidin-1- | 3.0 |
| | yl)benzene]sulfonyl}piperidine-3-carboxamide | |
| #27 | 1-{[4-(butylamino)benzene]sulfonyl}piperidine-4- | 9.3 |
| | carboxylic acid | |
| #28 | 3-bromo-5-(morpholin-4-yl)benzamide | 29.0 |
| #29 | 3-phenyl-1-(piperazin-1-yl)propan-1-one hydrochloride | 10.1 |
| #30 | 1-{4-[(4-fluorobenzene)sulfonyl]piperazin-1-yl}-3- | 1494.0 |
| | phenylpropan-1-one | |
| #31 | tert-butyl 4-{4-[4-(3-phenylpropanoyl)piperazine-1- | 3.0 |
| | sulfonyl]phenyl}piperazine-1-carboxylate | |
| #32 | 1-(4-{[4-(morpholin-4-yl)benzene]sulfonyl}piperazin-1- | 3.8 |
| | yl)-3-phenylpropan-1-one | |
| #33 | 1-(4-{[4-(cyclohexylamino)benzene]sulfonyl}piperazin- | 0.4 |
| | 1-yl)-3-phenylpropan-1-one | |
| #34 | 3-phenyl-1-(4-{[4-(piperidin-1- | 2.7 |
| | yl)benzene]sulfonyl}piperazin-1-yl)propan-1-one | |
| #35 | 3-phenyl-1-(4-{[4-(pyrrolidin-1- | 3.3 |
| | yl)benzene]sulfonyl}piperazin-1-yl)propan-1-one | |
| #36 | tert-butyl 4-(4-{3-[(2-phenylethyl)carbamoyl]piperidine- | 11.5 |
| | 1-sulfonyl}phenyl)piperazine-1-carboxylate | |
| #37 | N-(1-((4-ethoxyphenyl)sulfonyl)piperidin-3-yl)-3- | 28.5 |
| | phenylpropanamide | |
| #38 | N-(1-(4-(4-phenoxypiperidin-1- | 0.9 |
| | yl)phenylsulfonyl)piperidin-3-yl)-3-phenylpropanamide | |
| #39 | N-(1-(4-(4-isopropoxypiperidin-1- | 3.9 |
| | yl)phenylsulfonyl)piperidin-3-yl)-3-phenylpropanamide | |
| #40 | N-(1-(4-(4-methylpiperidin-1-yl)phenylsulfonyl)piperidin- | 0.8 |
| | 3-yl)-3-phenylpropanamide | |
| #41 | tert-butyl 1′-(4-(3-(3-phenylpropanamido)piperidin-1- | 1.8 |
| | ylsulfonyl)phenyl)-4,4′-bipiperidine-1-carboxylate | |
| #42 | 3-phenyl-N-(1-(4-(4-(trifluoromethyl)piperidin-1- | 0.9 |
| | yl)phenylsulfonyl)piperidin-3-yl)propanamide | |
| #43 | N-{1-[(3-chloro-4-fluorobenzene)sulfonyl]piperidin- | 3.0 |
| | 3-yl}-3-phenylpropanamide | |
| #44 | N-(1-{[4-(3-methylpiperidin-1- | 0.1 |
| | yl)benzene]sulfonyl}piperidin-3-yl)-3-phenylpropanamide | |
| #45 | N-(1-{[3-chloro-4-(piperidin-1- | 9.3 |
| | yl)benzene]sulfonyl}piperidin-3-yl)-3-phenylpropanamide | |
| #46 | N-(1-(4-(4,4-difluoropiperidin-1- | 3.8 |
| | yl)phenylsulfonyl)piperidin-3-yl)-3-phenylpropanamide | |
| #47 | N-(1-{[4-(2-methylpiperidin-1- | 0.1 |
| | yl)benzene]sulfonyl}piperidin-3-yl)-3-phenylpropanamide | |
| #48 | N-(3-phenylpropyl)-1-{[4-(piperidin-1- | 2.9 |
| | yl)benzene]sulfonyl}piperidin-3-amine | |
| #49 | N-{1-[4-(diisopropylsulfamoyl)phenyl]piperidin-3-yl}- | 2.6 |
| | 3-phenylpropanamide | |
| #50 | 3-phenyl-N-{1-[4-(phenylsulfamoyl)phenyl]piperidin-3- | 0.6 |
| | yl}propanamide | |
| #51 | N-{1-[4-(cyclohexylsulfamoyl)phenyl]piperidin-3-yl}- | 3.1 |
| | 3-phenylpropanamide | |
| #52 | N-(1-{4-[(furan-2-ylmethyl)sulfamoyl]phenyl}piperidin- | 2.2 |
| | 3-yl)-3-phenylpropanamide | |
| #53 | 3-phenyl-N-[1-(4-sulfamoylphenyl)piperidin-3- | 4.6 |
| | yl]propanamide | |
| #54 | 3-phenyl-N-{1-[4-(piperidine-1- | 1.5 |
| | sulfonyl)phenyl]piperidin-3-yl}propanamide | |
| #55 | 3-phenyl-N-{1-[4-(pyrrolidine-1- | 2.9 |
| | sulfonyl)phenyl]piperidin-3-yl}propanamide | |

TABLE 2
Effectiveness of inhibition for 5 μM of the compounds when
applied to P2X1R, P2X2R, P2X4R and P2X7R

| | Percentage of activity remains in the | | | | |
|---|---|---|---|---|---|
| | presence of the compound at 5 μM | | | | |
| | P2X1R | P2X2R | P2X2R | P2X4R | P2X7R |
| Compound | (Calcium | (Calcium | (Yo-Pro-1 | (Calcium | (Yo-Pro-1 |
| ID | Assay) | Assay) | Assay) | Assay) | Assay) |
| #1 | 56.5 | 61.3 | 44.0 | 71.9 | 20.0 |
| #2 | 39.1 | 85.7 | 54.6 | 56.4 | 15.8 |
| #3 | 33.3 | 98.0 | 57.0 | 67.0 | 13.5 |
| #4 | 43.7 | 83.7 | 64.9 | 56.9 | 38.4 |
| #5 | 59.6 | 87.8 | 55.5 | 53.1 | −7.3 |
| #6 | 51.0 | 55.2 | 54.3 | 98.9 | 10.7 |
| #7 | 65.6 | 78.8 | 77.6 | 76.8 | 14.1 |
| #8 | 67.0 | 94.6 | 63.8 | 50.9 | −14.5 |
| #9 | 92.8 | 61.3 | 76.9 | 84.5 | 10.2 |
| #10 | 51.5 | 69.3 | 38.2 | 69.5 | 70.1 |
| #11 | 44.3 | 52.0 | 44.0 | 59.6 | 80.9 |
| #12 | 52.8 | 64.8 | 41.8 | 69.1 | 44.3 |
| #13 | 54.2 | 67.4 | 48.9 | 88.8 | 0.8 |
| #14 | 63.9 | 72.7 | 54.3 | 70.9 | −6.1 |
| #15 | 70.1 | 80.4 | 85.0 | 63.8 | −12.2 |
| #16 | 51.5 | 77.0 | 83.8 | 60.7 | 26.0 |
| #17 | 60.9 | 64.9 | 91.6 | 77.6 | 53.4 |
| #18 | 62.3 | 73.1 | 52.7 | 61.6 | 41.5 |
| #19 | 54.4 | 97.0 | 50.3 | 75.1 | −11.2 |
| #20 | 75.4 | 68.1 | 61.5 | 50.6 | 7.0 |
| #21 | 50.2 | 94.3 | 50.8 | 31.0 | 36.2 |
| #22 | 59.2 | 85.8 | 72.0 | 42.1 | 43.0 |
| #23 | 75.5 | 57.7 | 77.1 | 70.7 | 35.8 |
| #24 | 66.3 | 73.5 | 70.9 | 68.1 | 32.6 |
| #25 | 66.1 | 70.0 | 79.3 | 61.0 | 36.8 |
| #26 | 60.5 | 103.2 | 68.5 | 67.3 | 0.3 |
| #27 | 50.8 | 67.2 | 55.4 | 59.9 | 59.0 |
| #28 | 62.6 | 50.7 | 77.3 | 85.4 | 75.2 |
| #29 | 62.5 | 83.3 | 89.4 | 67.1 | 88.1 |
| #30 | 70.1 | 67.5 | 74.9 | 76.8 | 52.2 |
| #31 | 43.9 | 78.3 | 64.0 | 81.8 | 12.9 |
| #32 | 46.7 | 47.8 | 79.9 | 91.0 | 54.3 |
| #33 | 44.9 | 83.6 | 60.9 | 83.9 | −8.5 |
| #34 | 59.3 | 65.2 | 72.4 | 53.8 | 4.8 |
| #35 | 56.3 | 43.7 | 70.6 | 70.7 | 23.6 |
| #36 | 49.0 | 49.4 | 61.8 | 72.2 | 47.3 |
| #37 | 55.4 | 90.3 | 58.1 | 54.8 | 82.7 |
| #38 | 43.8 | 52.2 | 57.6 | 71.1 | 7.5 |
| #39 | 74.5 | 86.6 | 66.3 | 68.2 | 11.1 |
| #40 | 39.0 | 73.5 | 69.1 | 62.9 | 9.1 |
| #41 | 46.7 | 61.1 | 61.4 | 39.9 | 18.3 |
| #42 | 66.3 | 62.7 | 59.2 | 55.8 | −1.7 |
| #43 | 57.1 | 56.0 | 71.1 | 48.4 | 23.5 |
| #44 | 40.5 | 83.0 | 61.4 | 63.5 | −5.1 |
| #45 | 52.0 | 49.4 | 83.3 | 61.9 | 33.1 |
| #46 | 67.7 | 71.0 | 39.1 | 75.0 | −8.6 |
| #47 | 50.1 | 65.6 | 69.7 | 93.3 | −7.4 |
| #48 | 56.4 | 48.1 | 74.5 | 65.5 | 10.4 |
| #49 | 65.8 | 82.6 | 55.9 | 35.3 | 1.4 |
| #50 | 47.8 | 67.2 | 59.7 | 84.3 | −1.1 |
| #51 | 63.1 | 81.2 | 46.6 | 71.3 | −1.0 |
| #52 | 63.4 | 72.3 | 45.8 | 52.8 | 36.9 |
| #53 | 66.2 | 58.0 | 70.9 | 49.7 | −0.5 |
| #54 | 48.4 | 59.4 | 81.7 | 63.3 | −9.3 |
| #55 | 53.8 | 52.7 | 85.4 | 37.7 | 4.6 |
| N Contl | −0.1 | 0.3 | 0.0 | −0.3 | 0.1 |
| P Contl | 52.4 | 93.9 | 100.2 | 67.8 | 91.5 |
| PPADS | 26.7 | 6.2 | 29.6 | N/A | 0.7 |

### Examples of the Invention were Found to be Ineffective to P2X1R Using an Assay Based on Receptor-Mediated Calcium Entrance (Calcium Assay)

Human P2X1 receptor (P2X1R) is a member of P2X family. Initial studies showed that the Yo-Pro-1 uptake assay was not useful for measuring P2X1R activity. Thus, the calcium assay was used. In the calcium assay, P2X receptor activation leads to opening of ion channels, which increases intracellular calcium concentration. The increased intracellular calcium concentration can be detected by a calcium indicator dye such as Fluo-4 or Fluo-8. Molecular Probes™ Fluo-4, AM (Cat. No. F14201 from Fisher Scientific) was used in the assay. The procedures were similar as described in the Yo-Pro-1 assay until day 4. The construct of P2X1R for transient transfection to HEK-293 cells was the courtesy from Dr. Richard J. Evans laboratory (J. Biol. Chem. 279:9043-9055). After transfection and expression, on day 4 cells were washed twice by Ca-HBSS (The composition (in mM) was NaCl 135, HEPES 10, D-glucose 10, KCl 5.4, CaCl₂ 2, and MgCl₂ 1, pH to 7.4 by NaOH) and incubated in dye loading solution (The solution contained 2.5 μM Fluo-4, 1 mM probenecid, and 1× PowerLoad (Cat. No. P10020 from Fisher Scientific) in Ca-HBSS) for 70 minutes at 37° C. Then the extracellular loading dye was washed away and cells were incubated at Ca-HBSS containing 1 mM probenecid for 30 minutes. Before the final steps, probenecid was washed away with Ca-HBSS. The assay was performed by adding 25 μl of Ca-HBSS followed by 25 μl of various test compounds. At this point, the base level of fluorescence was obtained through the microplate reading—the base microplate reading for this assay. At the end, the stimulus (ATP at 20 μM was the final concentration for P2X1R) was added to activate the P2X1R. Activation of P2X1R opened the channel to allow influx of calcium, which was represented by fluorescence increases that were measured by the microplate device with excitation at 475 nm and emission wavelength in the range of 500-550 nm. The data were analyzed the same way as described before: The result of each well was first normalized by its corresponding base microplate reading. Then, the negative control signal was subtracted from. In the negative control, the test compound and the stimulus were replaced by Ca-HBSS. The positive control was the same as the majority of the wells but did not contain any test compounds. An average of six highest values of the plate (AVG) were considered as 100% and the value of each well was divided by AVG and presented as the relative values in percentages. The results were listed in Table 2. At 5 μM, none of the compounds tested can completely inhibit P2X1R activity or reduce it to 15% of normal activity.

### Examples of the Invention were Found to be Ineffective to P2X2R Using an Assay Based on Yo-Pro-1 Uptake

Human P2X2 receptor (P2X2R) construct (J. Biol. Chem. 290, 7930-7942) was transiently transfected to HEK-293 cells and the Yo-Pro-1 assay was performed as described above. When 5 μM ATP was used as the stimulus, the elevation of receptor mediated activity can be seen in 10 minutes. The inhibitory results of the compounds of Formulas I-III tested to against P2X2R were listed in Table 2. At 5 μM, none of the compounds can completely inhibit P2X2R activity or reduce it to 15% of normal activity.

### Examples of the Invention were Found to be Ineffective to P2X2R Using an Assay Based on Receptor Mediated Calcium Entrance

When P2X2R was measured by the calcium assay as described above, the outcomes were essentially the same as from the Yo-Pro-1 assay. Their results were listed in Table 2.

### Examples of the Invention were Found to be Ineffective to P2X4R Using an Assay Based on Receptor Mediated Calcium Entrance

Human P2X4 receptor (P2X4R) construct (J. Biol. Chem. 290, 7930-7942) was transiently transfected to HEK-293 cells and the calcium assay was performed as described above. When 1 mM ATP was used as stimulus, an increased receptor mediated activity leading to intracellular calcium elevation can be seen in less than 10 minutes. The inhibitory results of the compounds of Formulas I-III to P2X4R were listed in Table 2. At 5 μM, none of the compounds can completely inhibit P2X4R activity or reduce it to 15% of normal activity.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

1. A compound having the following Formula I: 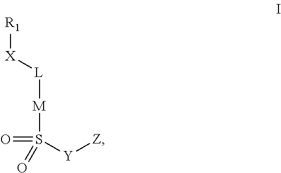
wherein:
R₁ is hydrogen, hydroxy, halogen, nitro, amino, alkyl, alkoxy, alkylamino, cycloalkyl, cycloalkyamino, heterocyclyl, aryl, heteroaryl, —NR₇R₈, —CO—R₁₀, or —NH—CO—R₁₀;
L is a bond, a heterocyclic bivalent group, a heteroaromatic bivalent group, or an aromatic bivalent group;
M is a bond, alkyl, aryl, a heterocyclic bivalent group, a heteroaromatic bivalent group, or an aromatic bivalent group;
X is a bond, —O—, —S—, —SO₂—, —CO—, —NR₉—, —(CH₂)ₘ—, or a heterocyclic bivalent group, m is 1, 2, 3, 4, 5, or 6;
Y is a bond, —NH—, a heterocyclic bivalent group, a heteroaromatic bivalent group, a bivalent benzyl group, or an aromatic bivalent group;
Z is hydrogen, halogen, alkyl, aryl, heterocyclyl, heteroaryl, —NR₇R₈, —CO—R₁₀, or —NH—CO—R₁₀;
R₇, R₈, and R₉ are independently hydrogen, hydroxy, halogen, nitro, amino, alkyl, alkoxy, alkylamino, cycloalkyl, cycloalkyamino, heterocyclyl, or heteroaryl; and
R₁₀ is —O-tert-butyl, —CH₂CH₂-phenyl, hydrogen, hydroxy, halogen, nitro, amino, alkyl, alkoxy, alkylamino, cycloalkyl, cycloalkyamino, heterocyclyl, or heteroaryl;
an isomer thereof, a tautomer thereof, a pharmaceutical acceptable solvate thereof, or a pharmaceutical acceptable prodrug thereof.

2. The compound of claim 1, wherein L is 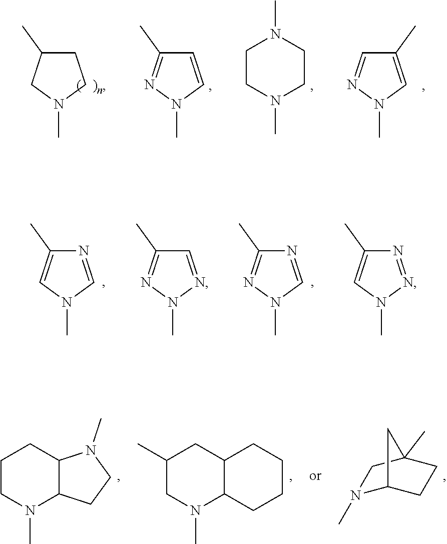
and n is 0, 1, 2, 3, 4, or 5.

3. The compound of claim 1, wherein Y is a bivalent phenyl group, a bivalent naphthyl group, a bivalent quinolinyl group, or a bivalent isoquinolinyl.

4. The compound of claim 1, wherein M in Formula I is a bond and the compound has the following Formula II: 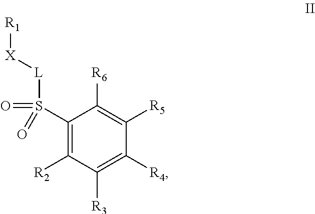
wherein:
R₁ is hydrogen, hydroxy, halogen, nitro, amino, alkyl, alkoxy, alkylamino, cycloalkyl, cycloalkyamino, heterocyclyl, aryl, heteroaryl, —NR₇R₈, —CO—R₁₀, or —NH—CO—R₁₀;
R₂, R₃, R₄, R₅, and R₆ are independently hydrogen, hydroxy, halogen, nitro, amino, alkyl, alkoxy, alkylamino, cycloalkyl, cycloalkyamino, heterocyclyl, or heteroaryl;
L is a bond, a heterocyclic bivalent group, a heteroaromatic bivalent group, or an aromatic bivalent group;
X is a bond, —O—, —S—, —SO₂—, —CO—, —NR₉—, or —(CH₂)ₘ—, m is 1, 2, 3, 4, 5, or 6;
R₇, R₈, and R₉ are independently hydrogen, hydroxy, halogen, nitro, amino, alkyl, alkoxy, alkylamino, cycloalkyl, cycloalkyamino, heterocyclyl, or heteroaryl; and
R₁₀ is —O-tert-butyl, —CH₂CH₂-phenyl, hydrogen, hydroxy, halogen, nitro, amino, alkyl, alkoxy, alkylamino, cycloalkyl, cycloalkyamino, heterocyclyl, or heteroaryl.

5. The compound of claim 4, wherein L is 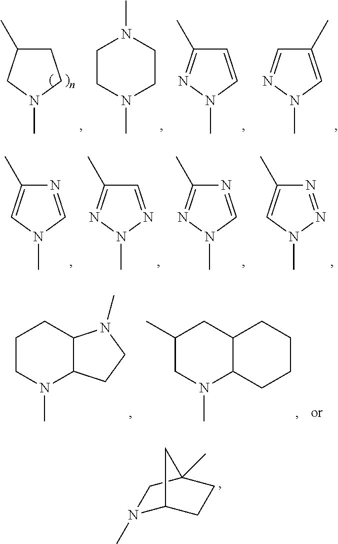
and n is 0, 1, 2, 3, 4, or 5.

6. The compound of claim 5, wherein R₂, R₃, R₄, R₅, and R₆ are independently hydrogen, hydroxy, halogen, nitro, amino, alkyl, alkoxy, alkylamino, cycloalkyl, cycloalkyamino, heteroaryl,

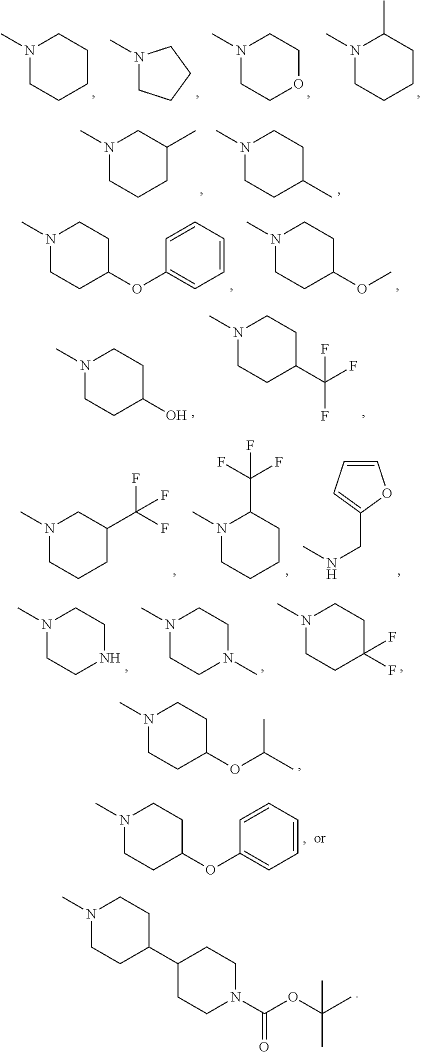

7. The compound of claim 6, wherein the compound is selected from the group consisting of:

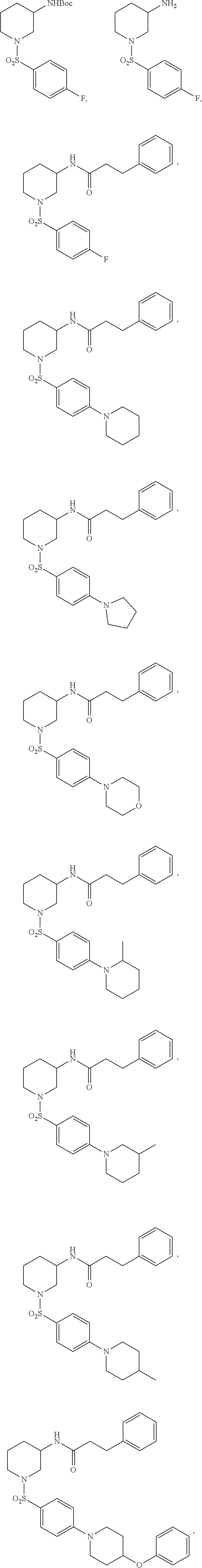

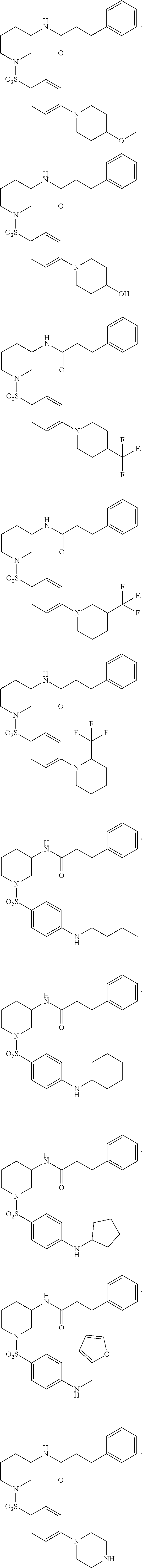

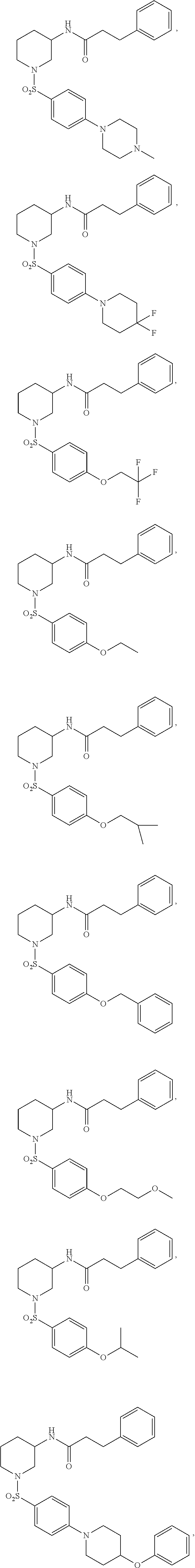

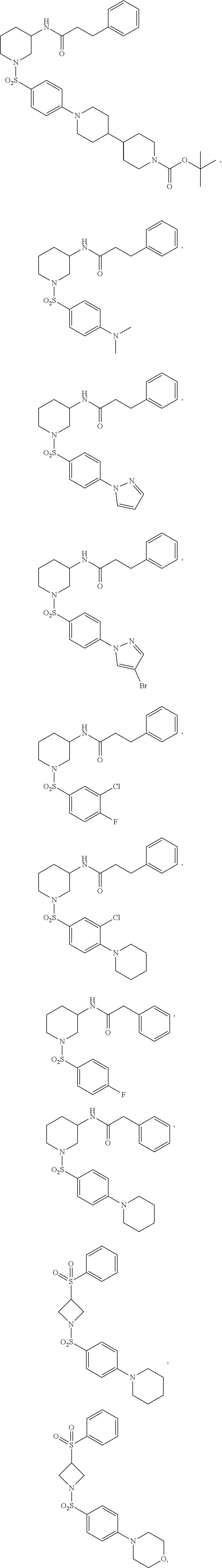

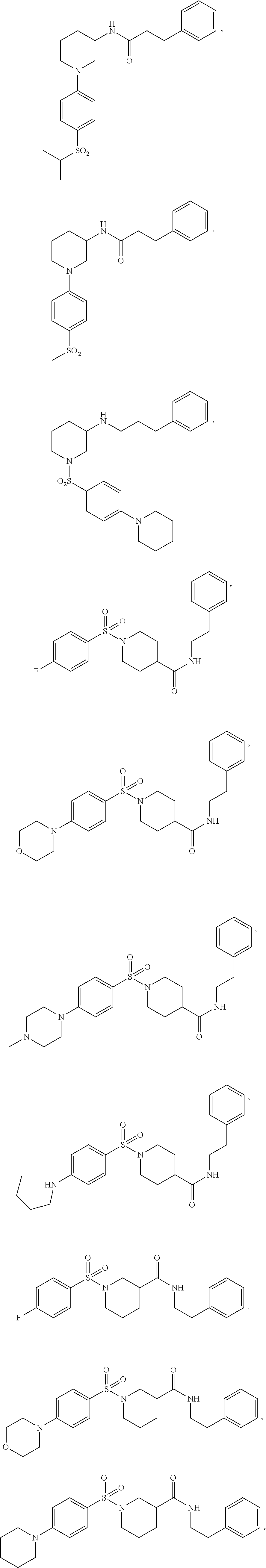

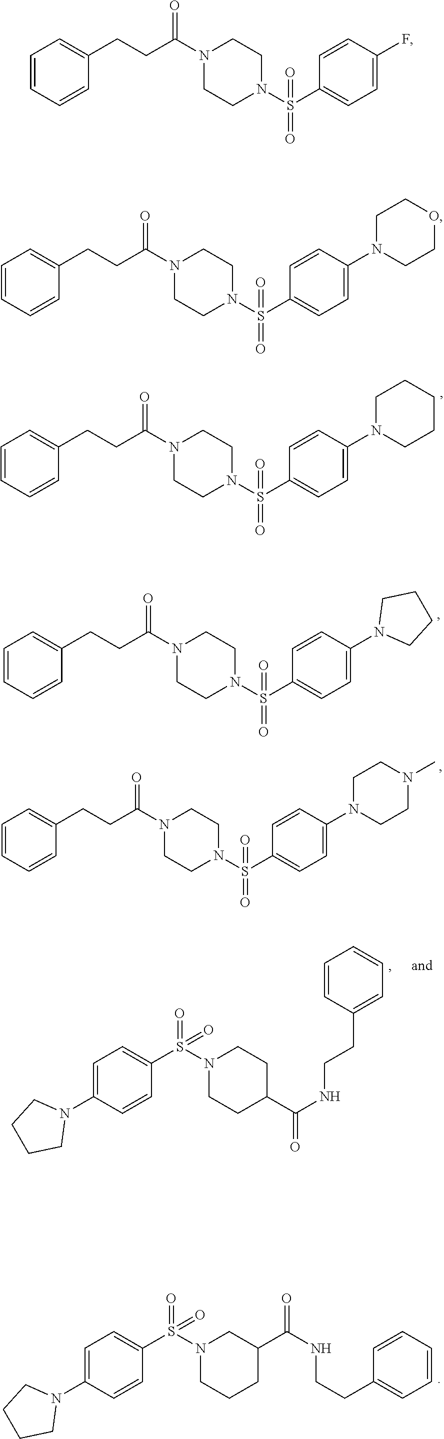

8. The compound of claim 1, wherein R₁—X-L- in Formula I is 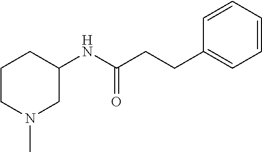 [SMILES: CN1CCCC(NC(=O)CCc2ccccc2)C1]
and the compound has the following Formula III: 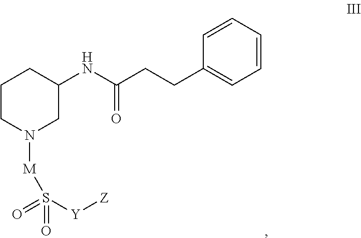
wherein:
M is a bond, alky, aryl, a heterocyclic bivalent group, a heteroaromatic bivalent group, or an aromatic bivalent group; and
Y is a bond, —NH—, a heterocyclic bivalent group, a heteroaromatic bivalent group, a bivalent benzyl group, or an aromatic bivalent group;
Z is hydrogen, halogen, alkyl, aryl, heterocyclyl, heteroaryl, —NR₇R₈, —CO—R₁₀, or —NH—CO—R₁₀;
R₇ and R₈ are independently hydrogen, hydroxy, halogen, nitro, amino, alkyl, alkoxy, alkylamino, cycloalkyl, cycloalkyamino, heterocyclyl, or heteroaryl; and
R₁₀ is —O-tert-butyl, —CH₂CH₂-phenyl, hydrogen, hydroxy, halogen, nitro, amino, alkyl, alkoxy, alkylamino, cycloalkyl, cycloalkyamino, heterocyclyl, or heteroaryl.

9. The compound of claim 8, wherein M is a bond; Y is a bivalent benzyl group; and Z is hydrogen or halogen.

10. The compound of claim 9, wherein the compound is selected from the group consisting of:

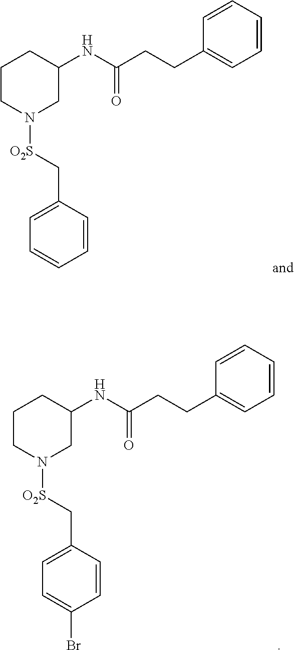 [SMILES: O=C(CCc1ccccc1)NC1CCCN(S(=O)(=O)Cc2ccccc2)C1 ; O=C(CCc1ccccc1)NC1CCCN(S(=O)(=O)Cc2ccc(Br)cc2)C1 ; "."]

11. The compound of claim 8, wherein M is a bivalent phenyl group; Y is a bond or —NH—; Z is hydrogen, heterocyclyl, heteroaryl, —NR₇R₈, —CO—R₁₀, or —NH—CO—R₁₀; R₇ and R₈ are independently hydrogen or alkyl; and R₁₀ is —O-tert-butyl, —CH₂CH₂-phenyl, hydrogen, hydroxy, halogen, nitro, amino, alkyl, alkoxy, alkylamino, cycloalkyl, cycloalkyamino, heterocyclyl, or heteroaryl.

12. The compound of claim 11, wherein the compound is selected from the group consisting of: 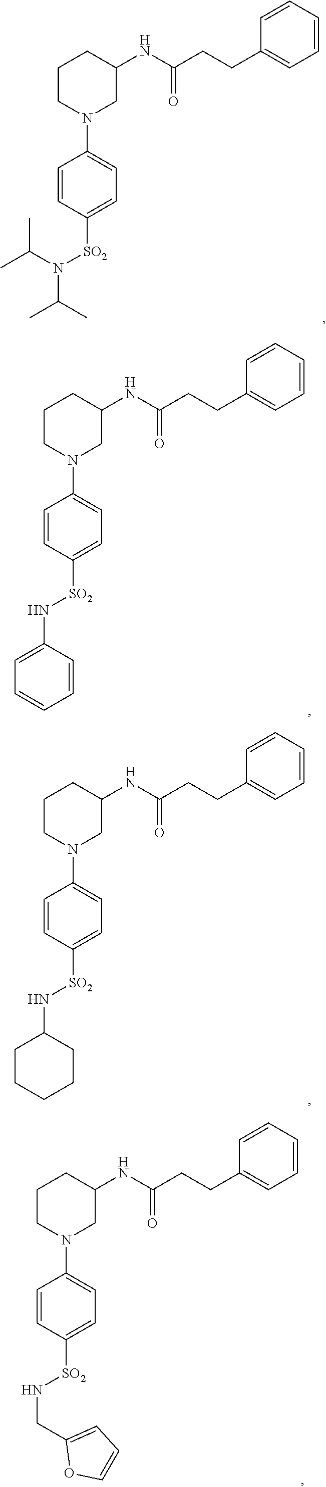 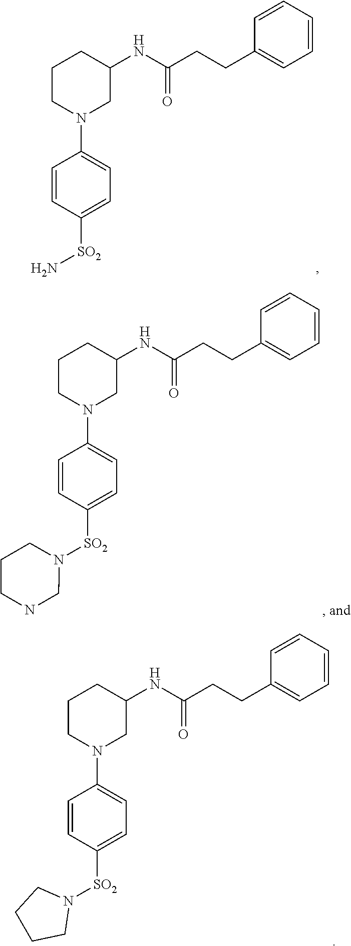 [SMILES: NS(=O)(=O)c1ccc(N2CCCC(NC(=O)CCc3ccccc3)C2)cc1 ; "," ; O=C(CCc1ccccc1)NC1CCCN(c2ccc(S(=O)(=O)N3CCCNC3)cc2)C1 ; ", and" ; O=C(CCc1ccccc1)NC1CCCN(c2ccc(S(=O)(=O)N3CCCC3)cc2)C1 ; "."]
